(19) United States
(12) Patent Application Publication
(10) Pub. No.: US 2021/0178040 A1
(43) Pub. Date: Jun. 17, 2021

(54) EXTRACORPOREAL CIRCULATION MANAGEMENT DEVICE, EXTRACORPOREAL CIRCULATION DEVICE, AND EXTRACORPOREAL CIRCULATION MANAGEMENT PROGRAM

(71) Applicant: TERUMO KABUSHIKI KAISHA, TOKYO (JP)

(72) Inventor: TOMOAKI HASHIMOTO, KANAGAWA (JP)

(21) Appl. No.: 17/188,143

(22) Filed: Mar. 1, 2021

Related U.S. Application Data
(63) Continuation of application No. PCT/JP2019/033967, filed on Aug. 29, 2019.

(30) Foreign Application Priority Data
Sep. 6, 2018 (JP) 2018-167079

(51) Int. Cl. A61M 1/16

(52) U.S. Cl. CPC A61M 1/1615; A61M 1/1698; A61M 2205/3334; A61M 2205/52; A61M 2205/3331; A61M 2205/502

(57) ABSTRACT

An extracorporeal circulation management device 10 displays an expected flow rate of blood flowing inside a circulation circuit 1R and an actual flow rate on a display unit 16, together with a standard pressure calculated based on the expected flow rate by referring to standard information representing a relation between the blood flow rate and a pressure loss occurring in each of devices 2, 5, and 6 provided in the circulation circuit 1R and stored in a storage unit, and an actual pressure related to each of the devices 2, 5, and 6, which is calculated based on an actual pressure measured by each of pressure measurement units 21, 22, and 23 as an actually measured value of a pressure of the blood flowing inside the circulation circuit 1R and the actual flow rate.

### CROSS REFERENCE TO RELATED APPLICATIONS

This application is a continuation of PCT Application No. PCT/JP2019/033967, filed Aug. 29, 2019, based on and claiming priority to Japanese Application No. 2018-167079, filed Sep. 6, 2018, both of which are incorporated herein by reference in their entirety.

### BACKGROUND OF THE INVENTION

The present invention relates to an extracorporeal circulation management device, an extracorporeal circulation device, and an extracorporeal circulation management program.

For example, when it is necessary to supply blood to a patient during a surgery, extracorporeal circulation is performed in which the patient's blood is circulated extracorporeally using an extracorporeal circulation device having an artificial heart-lung device or the like. In addition, there are various auxiliary circulation methods, such as veno-arterial extracorporeal membrane oxygenation (VA-ECMO) and veno-venous extracorporeal membrane oxygenation (VV-ECMO), as long-term life support by extracorporeal circulation of blood.

For example, in auxiliary circulation techniques using VV-ECMO, extracorporeal circulation has been increasingly performed for a long period of time. Therefore, it becomes important not only to manage a state of a patient but also to grasp states of devices such as circulation circuits, pumps, and oxygenators, used for extracorporeal circulation such that extracorporeal circulation can be appropriately performed.

In addition, use of devices, such as pressure sensors and flow rate sensors, extracorporeal circulation has increased. Accordingly, the number of items to be monitored in the auxiliary circulation techniques has increased. Therefore, it is required to understand the significance of values measured by the respective devices and to appropriately comprehend the values measured by the respective devices in the medical field. For example, it is important to pay attention to a distribution and a state of a pressure applied in a circulation circuit in the medical field. If a blood removal pressure (for example, a negative pressure) increases when a flow rate of blood flowing through the circulation circuit decreases, blood removal failure is suspected in the medical field based on a rule of thumb invoked by a device operator or the like. Further, the operator or the like inspects the circulation circuit to identify a cause of the blood removal failure in a circuit or device on the blood removal side, and resolves the blood removal failure.

Published Japanese patent application JP2017-38805A discloses an extracorporeal circulation management device that displays state information of a pressure sensor, a flow rate sensor, and the like on a display unit as continuous state information over time, and displays warning information on the display unit based on trend information of the continuous state information over time. In the extracorporeal circulation management device described in JP2017-38805A, however, there is room for improvement in that it may be difficult for an operator to easily recognize a cause of circulation failure in extracorporeal circulation although the continuous state information over time and the warning information are displayed on the display unit.

### SUMMARY OF THE INVENTION

The present invention has been made to solve the above problem, and an object thereof is to provide an extracorporeal circulation management device, an extracorporeal circulation device, and an extracorporeal circulation management program that allow an operator or like to easily recognize a cause and/or location of circulation failure in extracorporeal circulation.

According to the present invention, the above problem is solved by an extracorporeal circulation management device that manages an extracorporeal circulation device which extracorporeally circulates blood using a circulation circuit, the extracorporeal circulation management device displaying on a display unit an expected flow rate, which is assumed in advance (i.e., predicted using a model of nominal performance of the circulation device) as an expected value of a flow rate of the blood flowing inside the circulation circuit, and an actual flow rate. The actual flow rate is measured by a flow rate measurement unit as an actually measured value of the flow rate of the blood flowing inside the circulation circuit. The display unit also displays a standard pressure, which represents a relation between the blood flow rate and a pressure loss occurring in a device provided in the circulation circuit and is calculated based on the expected flow rate by referring to standard information stored in a storage unit, and an actual pressure related to the device, which is calculated based on an actual pressure measured by a pressure measurement unit as an actually measured value of a pressure of the blood flowing inside the circulation circuit and the actual flow rate, on the display unit.

According to the extracorporeal circulation management device of the present invention, the expected flow rate expected in advance as the expected value of the flow rate of the blood flowing inside the circulation circuit and the actual flow rate measured by the flow rate measurement unit as the actually measured value of the flow rate of the blood flowing inside the circulation circuit are displayed on the display unit. As a result, an operator and the like can easily grasp a discrepancy or deviation between the expected flow rate of the blood in the circulation circuit and the actual flow rate of the blood actually flowing through the circulation circuit by confirming the display unit. In addition, the standard pressure calculated based on the expected flow rate by referring to the standard information stored in the storage unit, which is the standard information representing the relation between the blood flow rate and the pressure loss occurring in the device provided in the circulation circuit under normal conditions, and the actual pressure related to the device, calculated based on the actual pressure measured by the pressure measurement unit as the actually measured value of the pressure of the blood flowing inside the circulation circuit and the actual flow rate measured by the flow rate measurement unit, are displayed on the display unit. As a result, the operator or the like can easily grasp a discrepancy or deviation between the standard pressure calculated based on the expected flow rate and the actual pressure related to the device by confirming the display unit. Therefore, when there is a discrepancy or deviation between the expected flow rate and the actual flow rate, the operator or the like can easily grasp the occurrence of the discrepancy or deviation between the expected flow rate and the actual flow rate and grasp a location of the circulation circuit where the discrepancy or deviation between the standard pressure and the actual pressure has occurred and easily grasp that a cause of circulation failure exists near the location by observing the display unit. That is, the location of the circulation circuit where the discrepancy or deviation between the standard pressure and the actual pressure has occurred corresponds to a portion where the pressure measurement unit is provided or a portion of the device provided near the pressure measurement unit. In this manner, the operator or the like can easily grasp the cause of the circulation failure in the extracorporeal circulation.

Preferably, the extracorporeal circulation management device according to the present invention further displays a differential flow rate representing a difference between the expected flow rate and the actual flow rate and a differential pressure representing a difference between the standard pressure and the actual pressure related to the device on the display unit.

According to the extracorporeal circulation management device of the present invention, the differential flow rate representing the difference between the expected flow rate and the actual flow rate and the differential pressure representing the difference between the standard pressure and the actual pressure related to the device are further displayed on the display unit. As a result, the operator or the like can more easily grasp a discrepancy or deviation between the expected flow rate and the actual flow rate and a discrepancy or deviation between the standard pressure and the actual pressure by confirming the display unit. The standard pressure is calculated based on the standard information stored in the storage unit and the expected flow rate. That is, the standard pressure changes depending on the expected flow rate. Therefore, a discrepancy or deviation sometimes occurs between the standard pressure and the actual pressure even if the actual pressure related to the device does not change at first glance. On the other hand, the operator or the like can more easily grasp the discrepancy or deviation between the standard pressure and the actual pressure by observing the display unit according to the extracorporeal circulation management device of the present invention.

Preferably, in the extracorporeal circulation management device according to the present invention, the device includes a plurality of instrument elements interconnected in the circulation circuit, and the pressure measurement unit includes a plurality of pressure sensors. A plurality of the standard pressures calculated based on the expected flow rate by referring to a plurality of pieces of the standard information each representing a relation between the blood flow rate and the pressure loss occurring in each of the plurality of instrument elements and stored in the storage unit, and the actual pressures related to the plurality of instrument elements, are calculated based on a plurality of the actual pressures measured by the plurality of pressure sensors and the actual flow rate, and then displayed on the display unit. A plurality of the differential pressures each representing a difference between each one of the plurality of standard pressures and each one of the actual pressures related to the plurality of instrument elements are displayed on the display unit.

According to the extracorporeal circulation management device of the present invention, the plurality of standard pressures calculated based on the expected flow rate by referring to the plurality of pieces of standard information stored in the storage unit (which are the plurality of pieces of standard information each representing the relation between the blood flow rate and the pressure loss occurring in each of the plurality of instrument elements) and the actual pressures related to the plurality of instrument elements are calculated based on the plurality of actual pressures measured by the plurality of pressure sensors and the actual flow rate measured by the flow rate measurement unit, and then displayed on the display unit. In addition, the plurality of differential pressures each representing the difference between each of the plurality of standard pressures and each of the actual pressures related to the plurality of instrument elements are displayed on the display unit, respectively. Therefore, the operator or the like can more easily grasp a discrepancy or deviation between each of the plurality of standard pressures and each of the actual pressures related to the plurality of instrument elements, and can more easily grasp a location in the circulation circuit where the discrepancy or deviation between the standard pressure and the actual pressure has occurred from among a plurality of locations and easily grasp that a cause of circulation failure exists near the location by observation of the display unit. That is, the location of the circulation circuit where the discrepancy or deviation between the standard pressure and the actual pressure has occurred corresponds to a portion where a pressure sensor detects an abnormal value among the plurality of pressure sensors is located or a portion of an instrument element provided near the pressure sensor detecting the abnormal value among the plurality of instrument elements is located.

Preferably, in the extracorporeal circulation management device according to the present invention, the plurality of instrument elements include: a blood removing catheter which is partially inserted into a patient and which guides the blood taken out from the patient; a pump which is provided on a downstream side of the blood removing catheter and which takes out the blood from the patient through the blood removing catheter and sends the blood to the downstream side; an oxygenator which is provided on the downstream side of the pump and performs a gas exchange operation for the blood; and a blood feeding catheter which is provided on the downstream side of the oxygenator and is partially inserted into the patient and which guides the blood that has passed through the oxygenator to the patient. A plurality of pressure sensors include: a first pressure sensor provided in the circulation circuit at least between the blood removing catheter and the pump or between the pump and the oxygenator; and a second pressure sensor provided in the circulation circuit between the oxygenator and the blood feeding catheter. A flow rate measurement unit is a flow rate sensor provided in the circulation circuit. All the 1) expected flow rate, the actual flow rate acquired by the flow rate sensor, 2) the differential flow rate, 3) the standard pressure related to the blood removing catheter calculated based on the expected flow rate by referring to the standard information related to the blood removing catheter, which represents a relation between the blood flow rate and the pressure loss occurring in the blood removing catheter and is stored in the storage unit, 4) the standard pressure related to the oxygenator calculated based on the expected flow rate by referring to the standard information related to the oxygenator, which represents a relation between the blood flow rate and the pressure loss occurring in the oxygenator and is stored in the storage unit, 5) the standard pressure related to the blood feeding catheter calculated based on the expected flow rate by referring to the standard information related to the blood feeding catheter, which represents a relation between the blood flow rate and the pressure loss occurring in the blood feeding catheter and is stored in the storage unit, 6) the actual pressure related to the blood removing catheter calculated based on the actual pressure measured by the first pressure sensor and the actual flow rate, 7) the actual pressure related to the oxygenator calculated based on the actual pressure measured by the first pressure sensor, the actual pressure measured by the second pressure sensor, and the actual flow rate, 8) the actual pressure related to the blood feeding catheter calculated based on the actual pressure measured by the second pressure sensor and the actual flow rate, the differential pressure related to the blood removing catheter which represents a difference between the standard pressure related to the blood removing catheter and the actual pressure related to the blood removing catheter, 9) the differential pressure related to the oxygenator which represents a difference between the standard pressure related to the oxygenator and the actual pressure related to the oxygenator, and 10) the differential pressure related to the blood feeding catheter which represents a difference between the standard pressure related to the blood feeding catheter and the actual pressure related to the blood feeding catheter are simultaneously displayed on the display unit.

According to the extracorporeal circulation management device of the present invention, the standard pressure related to the blood removing catheter calculated based on the expected flow rate by referring to the standard information related to the blood removing catheter stored in the storage unit, which is the standard information related to the blood removing catheter representing the relation between the blood flow rate and the pressure loss occurring in the blood removing catheter, is displayed on the display unit. In addition, the standard pressure related to the oxygenator calculated based on the expected flow rate by referring to the standard information related to the oxygenator stored in the storage unit, which is the standard information related to the oxygenator representing the relation between the blood flow rate and the pressure loss occurring in the oxygenator, is displayed on the display unit. In addition, the standard pressure related to the blood feeding catheter calculated based on the expected flow rate by referring to the standard information related to the blood feeding catheter stored in the storage unit, which is the standard information related to the blood feeding catheter representing the relation between the blood flow rate and the pressure loss occurring in the blood feeding catheter, is displayed on the display unit. In addition, the actual pressure related to the blood removing catheter, calculated based on the actual pressure measured by the first pressure sensor and the actual flow rate acquired by the flow rate sensor, is displayed on the display unit. In addition, the actual pressure related to the oxygenator, calculated based on the actual pressure measured by the first pressure sensor, the actual pressure measured by the second pressure sensor, and the actual flow rate acquired by the flow rate sensor, is displayed on the display unit. In addition, the actual pressure related to the blood feeding catheter, calculated based on the actual pressure measured by the second pressure sensor and the actual flow rate acquired by the flow rate sensor, is displayed on the display unit. In addition, the differential pressure related to the blood removing catheter, which represents the difference between the standard pressure related to the blood removing catheter and the previous pressure related to the blood removing catheter, is displayed on the display unit. In addition, the differential pressure related to the oxygenator, which represents the difference between the standard pressure related to the oxygenator and the actual pressure related to the oxygenator, is displayed on the display unit. In addition, the differential pressure related to the blood feeding catheter, which represents the difference between the standard pressure related to the blood feeding catheter and the actual pressure related to the blood feeding catheter, is displayed on the display unit. Further, all the expected flow rate, the actual flow rate, the differential flow rate, the standard pressure related to the blood removing catheter, the standard pressure related to the oxygenator, the standard pressure related to the blood feeding catheter, the actual pressure related to the blood removing catheter, the actual pressure related to the oxygenator, the actual pressure related to the blood feeding catheter, the differential pressure related to the blood removing catheter, the differential pressure related to the oxygenator, and the differential pressure related to the blood feeding catheter are simultaneously displayed on the display unit. As a result, the operator or the like can easily grasp a discrepancy or deviation between the standard pressure and the actual pressure and more concretely and easily grasp a location of the circulation circuit where the discrepancy or deviation between the standard pressure and the actual pressure has occurred and easily grasp that a cause of circulation failure exists near the location by observing the display unit. That is, the location of the circulation circuit where the discrepancy or deviation between the standard pressure and the actual pressure has occurred corresponds to a portion where a pressure sensor detecting an abnormal value between the first pressure sensor and the second pressure sensor is provided or a portion of an instrument element provided near the pressure sensor detecting the abnormal value among the blood removing catheter, the oxygenator, and the blood feeding catheter.

Preferably, in the extracorporeal circulation management device according to the present invention, the plurality of instrument elements interconnected in a circulation circuit include: a blood removing catheter which is partially inserted into a patient and guides the blood taken out from the patient; a pump which is provided on a downstream side of the blood removing catheter, takes out the blood from the patient through the blood removing catheter, and sends the blood to the downstream side; an oxygenator which is provided on the downstream side of the pump and performs a gas exchange operation for the blood; and a blood feeding catheter which is provided on the downstream side of the oxygenator and is partially inserted into the patient, and which guides the blood that has passed through the oxygenator to the patient. A plurality of pressure sensors include: a first pressure sensor provided in the circulation circuit between the blood removing catheter and the pump; a second pressure sensor provided in the circulation circuit between the pump and the oxygenator; and a third pressure sensor provided in the circulation circuit between the oxygenator and the blood feeding catheter. The flow rate measurement unit is a flow rate sensor provided in the circulation circuit. All the expected flow rate, the actual flow rate acquired by the flow rate sensor, the differential flow rate, the standard pressure related to the blood removing catheter calculated based on the expected flow rate by referring to the standard information related to the blood removing catheter which represents a relation between the blood flow rate and the pressure loss occurring in the blood removing catheter and is stored in the storage unit, the standard pressure related to the oxygenator calculated based on the expected flow rate by referring to the standard information related to the oxygenator which represents a relation between the blood flow rate and the pressure loss occurring in the oxygenator and is stored in the storage unit, the standard pressure related to the blood feeding catheter calculated based on the expected flow rate by referring to the standard information related to the blood feeding catheter which represents a relation between the blood flow rate and the pressure loss occurring in the blood feeding catheter and is stored in the storage unit, the actual pressure related to the blood removing catheter calculated based on the actual pressure measured by the first pressure sensor and the actual flow rate, the actual pressure related to the oxygenator calculated based on the actual pressure measured by the second pressure sensor, the actual pressure measured by the third pressure sensor, and the actual flow rate, the actual pressure related to the blood feeding catheter calculated based on the actual pressure measured by the third pressure sensor and the actual flow rate, the differential pressure related to the blood removing catheter which represents a difference between the standard pressure related to the blood removing catheter and the actual pressure related to the blood removing catheter, the differential pressure related to the oxygenator which represents a difference between the standard pressure related to the oxygenator and the actual pressure related to the oxygenator, and the differential pressure related to the blood feeding catheter which represents a difference between the standard pressure related to the blood feeding catheter and the actual pressure related to the blood feeding catheter are simultaneously displayed on the display unit.

According to the extracorporeal circulation management device of the present invention, the standard pressure related to the blood removing catheter calculated based on the expected flow rate by referring to the standard information related to the blood removing catheter stored in the storage unit, which is the standard information related to the blood removing catheter representing the relation between the blood flow rate and the pressure loss occurring in the blood removing catheter, is displayed on the display unit. In addition, the standard pressure related to the oxygenator calculated based on the expected flow rate by referring to the standard information related to the oxygenator stored in the storage unit, which is the standard information related to the oxygenator representing the relation between the blood flow rate and the pressure loss occurring in the oxygenator, is displayed on the display unit. In addition, the standard pressure related to the blood feeding catheter calculated based on the expected flow rate by referring to the standard information related to the blood feeding catheter stored in the storage unit, which is the standard information related to the blood feeding catheter representing the relation between the blood flow rate and the pressure loss occurring in the blood feeding catheter, is displayed on the display unit. In addition, the actual pressure related to the blood removing catheter, calculated based on the actual pressure measured by the first pressure sensor and the actual flow rate acquired by the flow rate sensor, is displayed on the display unit. In addition, the actual pressure related to the oxygenator, calculated based on the actual pressure measured by the second pressure sensor, the actual pressure measured by the third pressure sensor, and the actual flow rate acquired by the flow rate sensor, is displayed on the display unit. In addition, the actual pressure related to the blood feeding catheter, calculated based on the actual pressure measured by the third pressure sensor and the actual flow rate acquired by the flow rate sensor, is displayed on the display unit. In addition, the differential pressure related to the blood removing catheter, which represents the difference between the standard pressure related to the blood removing catheter and the actual pressure related to the blood removing catheter, is displayed on the display unit. In addition, the differential pressure related to the oxygenator, which represents the difference between the standard pressure related to the oxygenator and the actual pressure related to the oxygenator, is displayed on the display unit. In addition, the differential pressure related to the blood feeding catheter, which represents the difference between the standard pressure related to the blood feeding catheter and the actual pressure related to the blood feeding catheter, is displayed on the display unit. Further, all the expected flow rate, the actual flow rate, the differential flow rate, the standard pressure related to the blood removing catheter, the standard pressure related to the oxygenator, the standard pressure related to the blood feeding catheter, the actual pressure related to the blood removing catheter, the actual pressure related to the oxygenator, the actual pressure related to the blood feeding catheter, the differential pressure related to the blood removing catheter, the differential pressure related to the oxygenator, and the differential pressure related to the blood feeding catheter are simultaneously displayed on the display unit. As a result, the operator or the like can easily grasp a discrepancy or deviation between the standard pressure and the actual pressure and more concretely and easily grasp a location of the circulation circuit where the discrepancy or deviation between the standard pressure and the actual pressure has occurred and easily grasp that a cause of circulation failure exists near the location by observing the display unit. That is, the location of the circulation circuit where the discrepancy or deviation between the standard pressure and the actual pressure has occurred corresponds to a portion where a pressure sensor detecting an abnormal value among the first pressure sensor, the second pressure sensor, and the third pressure sensor is provided or a portion of an instrument element provided near the pressure sensor detecting the abnormal value among the blood removing catheter, the oxygenator, and the blood feeding catheter.

Preferably, the extracorporeal circulation management device according to the present invention provides notification of a warning when an absolute value of at least one of the differential flow rate and the differential pressure exceeds a predetermined value.

According to the extracorporeal circulation management device of the present invention, the operator or the like can more easily grasp occurrence of a discrepancy or deviation between the expected flow rate and the actual flow rate and further grasp a location of the circulation circuit where the discrepancy or deviation between the standard pressure and the actual pressure has occurred and more easily grasp that a cause of circulation failure exists near the location.

According to the present invention, the above problem is solved by an extracorporeal circulation device which extracorporeally circulates blood using a circulation circuit, the extracorporeal circulation device including: a display unit that displays various types of information; a blood removing catheter which is partially inserted into a patient and guides the blood taken out from the patient; a pump which is provided on a downstream side of the blood removing catheter, takes out the blood from the patient through the blood removing catheter, and sends the blood to the downstream side; an oxygenator which is provided on the downstream side of the pump and performs a gas exchange operation for the blood; a blood feeding catheter which is provided on the downstream side of the oxygenator, is partially inserted into the patient, and guides the blood that has passed through the oxygenator to the patient; and any of the above-described extracorporeal circulation management devices.

According to the extracorporeal circulation device of the present invention, the assumed flow rate assumed (i.e., predicted using a model of nominal performance of the circulation device) in advance as the expected value of the flow rate of the blood flowing inside the circulation circuit and the actual flow rate measured by the flow rate measurement unit as the actually measured value of the flow rate of the blood flowing inside the circulation circuit are displayed on the display unit. As a result, an operator and the like can easily grasp a discrepancy or deviation between the expected flow rate of the blood in the circulation circuit and the actual flow rate of the blood actually flowing through the circulation circuit by confirming the display unit. In addition, the standard pressure calculated based on the expected flow rate by referring to the standard information stored in the storage unit, which is the standard information representing the relation between the blood flow rate and the pressure loss occurring in the device, such as the blood removing catheter, the pump, the oxygenator, and the blood feeding catheter, provided in the circulation circuit, and the actual pressure related to the device, calculated based on the actual pressure measured by the pressure measurement unit as the actually measured value of the pressure of the blood flowing inside the circulation circuit and the actual flow rate measured by the flow rate measurement unit, are displayed on the display unit. As a result, the operator or the like can easily grasp a discrepancy or deviation between the standard pressure calculated based on the expected flow rate and the actual pressure related to the device by observing the display unit. Therefore, when there is a discrepancy or deviation between the expected flow rate and the actual flow rate, the operator or the like can easily grasp the occurrence of the discrepancy or deviation between the expected flow rate and the actual flow rate and grasp a location of the circulation circuit where the discrepancy or deviation between the standard pressure and the actual pressure has occurred and easily grasp that a cause of circulation failure exists near the location by confirming the display unit. That is, the location of the circulation circuit where the discrepancy or deviation between the standard pressure and the actual pressure has occurred corresponds to a portion where the pressure measurement unit is provided or a portion of the device provided near the pressure measurement unit. In this manner, the operator or the like can easily grasp the cause of the circulation failure in the extracorporeal circulation.

According to the present invention, the above problem is solved by an extracorporeal circulation management program executed by a computer of an extracorporeal circulation management device that manages an extracorporeal circulation device which extracorporeally circulates blood using a circulation circuit, the extracorporeal circulation management program causing the computer to execute a step of displaying an expected flow rate, which is predicted in advance as an expected value of a flow rate of the blood flowing inside the circulation circuit, and an actual flow rate, which is measured by a flow rate measurement unit as an actually measured value of the flow rate of the blood flowing inside the circulation circuit, on a display unit, and displaying a standard pressure calculated based on the expected flow rate by referring to standard information, which represents a relation between the blood flow rate and a pressure loss occurring in a device provided in the circulation circuit and is stored in a storage unit, and an actual pressure related to the device, which is calculated based on an actual pressure measured by a pressure measurement unit as an actually measured value of a pressure of the blood flowing inside the circulation circuit and the actual flow rate, on the display unit.

According to the extracorporeal circulation management program of the present invention, the expected flow rate predicted in advance as the expected value of the flow rate of the blood flowing inside the circulation circuit and the actual flow rate measured by the flow rate measurement unit as the actually measured value of the flow rate of the blood flowing inside the circulation circuit are displayed on the display unit. As a result, an operator and the like can easily grasp a discrepancy or deviation between the expected flow rate of the blood in the circulation circuit and the actual flow rate of the blood actually flowing through the circulation circuit by confirming the display unit. In addition, the standard pressure calculated based on the expected flow rate by referring to the standard information stored in the storage unit, which is the standard information representing the relation between the blood flow rate and the pressure loss occurring in the device provided in the circulation circuit, and the actual pressure related to the device, calculated based on the actual pressure measured by the pressure measurement unit as the actually measured value of the pressure of the blood flowing inside the circulation circuit and the actual flow rate measured by the flow rate measurement unit, are displayed on the display unit. As a result, the operator or the like can easily grasp a discrepancy or deviation between the standard pressure calculated based on the expected flow rate and the actual pressure related to the device by observing the display unit. Therefore, when there is a discrepancy or deviation between the expected flow rate and the actual flow rate, the operator or the like can easily grasp the occurrence of the discrepancy or deviation between the expected flow rate and the actual flow rate and grasp a location of the circulation circuit where the discrepancy or deviation between the standard pressure and the actual pressure has occurred and easily grasp that a cause of circulation failure exists near the location by confirming the display unit. That is, the location of the circulation circuit where the discrepancy or deviation between the standard pressure and the actual pressure has occurred corresponds to a portion where the pressure measurement unit is provided or a portion of the device provided near the pressure measurement unit. In this manner, the operator or the like can easily grasp the cause of the circulation failure in the extracorporeal circulation.

According to the present invention, it is possible to provide the extracorporeal circulation management device, the extracorporeal circulation device, and the extracorporeal circulation management program that allow the operator or the like to easily grasp the cause of the circulation failure in the extracorporeal circulation.

### BRIEF DESCRIPTION OF THE DRAWINGS

FIG. 1 is a schematic diagram illustrating an extracorporeal circulation device according to an embodiment of the present invention.

FIG. 2 is a schematic diagram illustrating an extracorporeal circulation device according to a modification of the present embodiment.

FIG. 3 is a block diagram illustrating a main configuration of an extracorporeal circulation management device according to the present embodiment.

FIG. 4 is a block diagram illustrating a main configuration of the extracorporeal circulation device according to the present embodiment.

FIG. 5 is a schematic diagram illustrating a standard information storage unit of the present embodiment.

FIG. 6 is a graph illustrating an example of standard information related to a blood removing catheter of the present embodiment.

FIG. 7 is a graph illustrating an example of standard information related to an oxygenator of the present embodiment.

FIG. 8 is a graph illustrating an example of standard information related to a blood feeding catheter of the present embodiment.

FIG. 9 is a schematic diagram illustrating a standard pressure calculation unit of the present embodiment.

FIG. 10 is a schematic diagram illustrating an actual pressure calculation unit of the present embodiment.

FIG. 11 is a schematic diagram illustrating a differential pressure calculation unit of the present embodiment.

FIG. 12 is a schematic diagram illustrating a warning information storage unit of the present embodiment.

FIG. 13 is a schematic diagram illustrating an example of an image displayed on an external monitor according to the present embodiment.

FIG. 14 is a schematic diagram illustrating an example of a relation between arrangements of a circulation circuit and each device of the extracorporeal circulation device according to the present embodiment and a pressure loss.

FIG. 15 is a schematic diagram illustrating another example of an image displayed on an external monitor of the present embodiment.

FIG. 16 is a flowchart illustrating an example of control executed by a computer of the extracorporeal circulation management device according to the present embodiment.

FIG. 17 is a flowchart illustrating an example of the control executed by the computer of the extracorporeal circulation management device according to the present embodiment.

### DETAILED DESCRIPTION OF PREFERRED EMBODIMENTS

Hereinafter, preferred embodiments of the present invention will be described in detail with reference to the drawings. The embodiments to be described below are preferable specific examples of the present invention, and thus, various technically preferable limitations are given. However, a scope of the present invention is not limited to these aspects as long as there is no particular description to limit the present invention in the following description. In addition, the same components will be denoted by the same reference signs in the respective drawings, and the detailed description thereof will be omitted as appropriate.

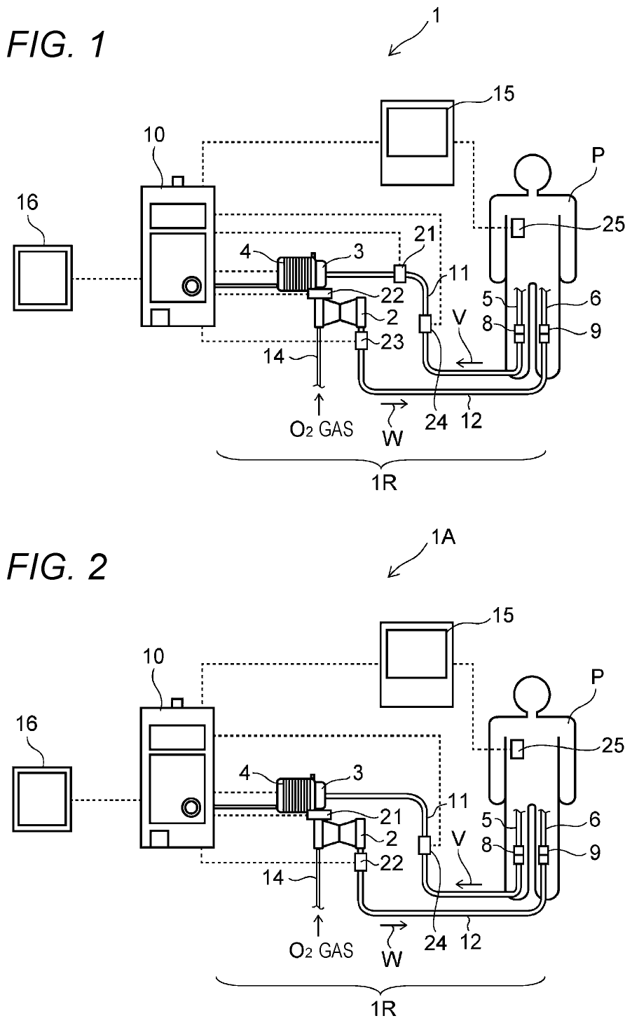

FIG. 1 is a schematic diagram illustrating an extracorporeal circulation device according to an embodiment of the present invention. Incidentally, an electrical connection is indicated by a broken line in FIG. 1. The electrical connection may be realized by a wire or wirelessly realized.

“Extracorporeal circulation” performed by an extracorporeal circulation device 1 illustrated in FIG. 1 includes an “extracorporeal circulation operation” and an “auxiliary circulation operation”. The extracorporeal circulation device 1 can perform both the “extracorporeal circulation operation” and the “auxiliary circulation operation”.

The “extracorporeal circulation operation” refers to a blood circulation operation and a gas exchange operation (oxygen addition and/or carbon dioxide removal) for blood performed by the extracorporeal circulation device 1, for example, in a case where blood circulation in the heart is temporarily stopped due to a cardiac surgery. In addition, the “auxiliary circulation operation” refers to a blood circulation operation and a gas exchange operation for blood that are also performed by the extracorporeal circulation device 1 in a case where the heart of a patient P to which the extracorporeal circulation device 1 is applied, hardly exhibits a sufficient function or in a state where it is difficult to sufficiently perform gas exchange by lungs.

For example, the extracorporeal circulation device 1 is applied in a case where the heart of the patient P does not operate normally or a case where the heart of the patient P operates normally but the lungs do not operate normally. Meanwhile, the extracorporeal circulation device 1 illustrated in FIG. 1 is used, for example, in a case of performing a cardiac surgery on the patient P or in subsequent treatment in an ICU. The extracorporeal circulation device 1 illustrated in FIG. 1 can operate a pump of the extracorporeal circulation device 1 to remove blood from the patient's vein and exchange a gas in the blood using an oxygenator to oxygenate the blood, and then, perform extracorporeal blood circulation of the oxygenator to return the oxygenated blood back to the patient's artery or vein. In this manner, the extracorporeal circulation device 1 is a device that acts as a substitute for the heart and lungs.

As illustrated in FIG. 1, the extracorporeal circulation device 1 has a circulation circuit 1R that circulates blood. The circulation circuit 1R includes an oxygenator 2, a centrifugal pump 3, a drive motor 4 that drives the centrifugal pump 3, a venous catheter (blood removing catheter) 5, an arterial catheter (blood feeding catheter) 6, and an extracorporeal circulation management device 10. The venous catheter 5 is an example of a “blood removing catheter” of the present invention. The arterial catheter 6 is an example of a “blood feeding catheter” of the present invention. The centrifugal pump 3 of the present embodiment is an example of an “instrument element” included in a “device” of the present invention. The extracorporeal circulation management device 10 is provided as a controller of the extracorporeal circulation device 1. Incidentally, the centrifugal pump 3 is also called a blood pump or the like, and may be a pump other than the centrifugal type.

The blood removing catheter 5 is also called a venous cannula (blood removing cannula) and is inserted from the femoral vein. A distal end of the blood removing catheter 5 is placed in the right atrium. The blood removing catheter 5 is connected to a blood removal tube (also referred to as a blood removal line) 11 through a connector 8, is connected to the centrifugal pump 3 through the blood removal tube 11, and guides blood taken out from the patient P to the centrifugal pump 3 through the blood removal tube 11. The blood removal tube 11 is a conduit that connects the blood removing catheter 5 and the centrifugal pump 3, and is the conduit guiding the blood taken out from the patient P through the blood removing catheter 5 to the centrifugal pump 3. The blood removing catheter 5 of the present embodiment is an example of the “instrument element” included in the “device” of the present invention.

The blood feeding catheter 6 is also called an arterial cannula (blood feeding cannula) and is inserted from the femoral artery. The blood feeding catheter 6 is connected to a blood feeding tube (also referred to as a blood feeding line) 12 through the connector 9, is also connected to the oxygenator 2 through the blood feeding tube 12, and guides the blood that has passed through the oxygenator 2 to the patient P through the blood feeding tube 12. The blood feeding tube 12 is a conduit that connects the oxygenator 2 and the blood feeding catheter 6, and is the conduit guiding the blood having passed through the oxygenator 2 to the patient P. The blood feeding catheter 6 of the present embodiment is an example of the “instrument element” included in the “device” of the present invention.

The drive motor 4 controls driving of the centrifugal pump 3 based on a command of the extracorporeal circulation management device 10. The centrifugal pump 3 is provided on the downstream side of the blood removing catheter 5, and is driven by receiving a driving force transmitted from the drive motor 4. The centrifugal pump 3 takes out blood from the patient P through the blood removing catheter 5 and the blood removal tube 11, sends the blood to the oxygenator 2, and then, returns the blood to the patient P through the blood feeding tube 12.

The oxygenator 2 is provided on the downstream side of the centrifugal pump 3. Specifically, the oxygenator 2 is arranged between the centrifugal pump 3 and the blood feeding tube 12. The oxygenator 2 performs the gas exchange operation (oxygen addition and/or carbon dioxide removal) for blood. The oxygenator 2 is, for example, a membrane oxygenator, but is particularly preferably a hollow fiber membrane oxygenator. An oxygen gas is supplied to the oxygenator 2 through an oxygen supply tube 14. The oxygenator 2 of the present embodiment is an example of an “instrument element” included in a “device” of the present invention.

As the blood removal tube 11 and the blood feeding tube 12, for example, a conduit made of a synthetic resin which is highly transparent, elastically deformable, and flexible, such as a vinyl chloride resin and silicone rubber, is used. Blood, which is a liquid, flows in a V direction in the blood removal tube 11 and flows in a W direction in the blood feeding tube 12.

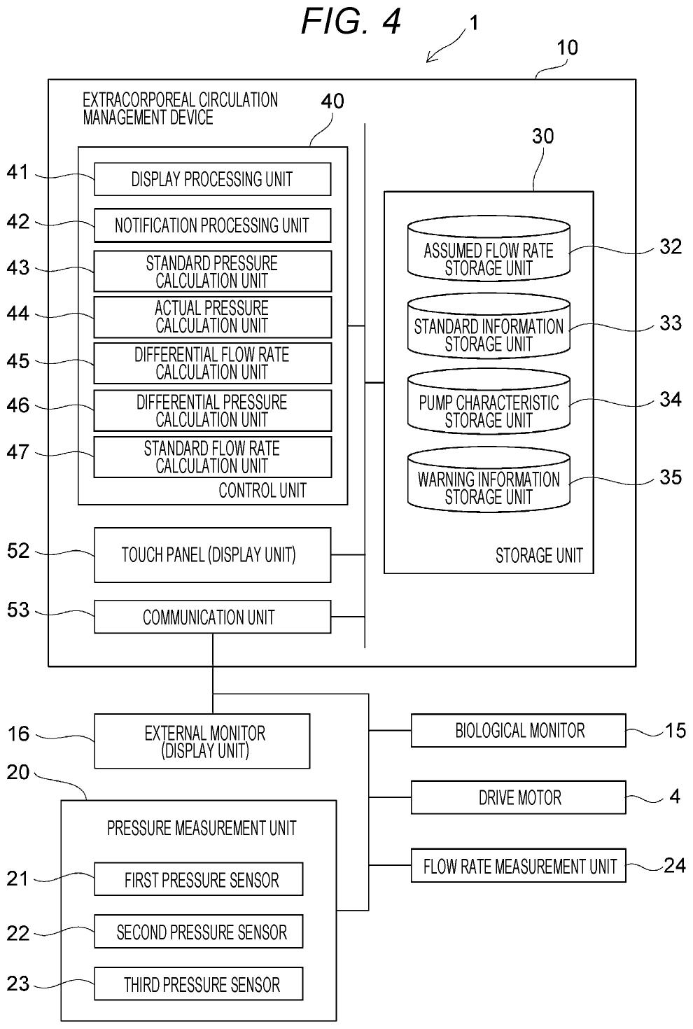

The extracorporeal circulation management device 10 acquires various types of information to perform a calculation, generates a control signal to control operations of devices such as the drive motor 4, a biological monitor 15, and an external monitor 16, and transmits the generated control signal to each device. In other words, the extracorporeal circulation management device 10 manages the extracorporeal circulation device 1. Details of the extracorporeal circulation management device 10 will be described later. In addition, the extracorporeal circulation management device 10 may have a touch panel 52 (see FIG. 4) as an input unit capable of inputting various types of information and as a display unit displaying the various types of information. That is, the “display unit” of the present invention may be the external monitor 16 provided as a separate body from the extracorporeal circulation management device 10, or may be the touch panel 52 provided in the extracorporeal circulation management device 10. The touch panel 52 is capable of detecting contact or the like of a finger of an operator or the like.

The extracorporeal circulation device 1 according to the present embodiment further includes a pressure measurement unit 20 (see FIG. 4), a flow rate measurement unit 24, a blood pressure measurement unit 25, the biological monitor 15, and the external monitor (display unit) 16. The external monitor 16 of the present embodiment is an example of the “display unit” of the present invention. In the following description, a case where the “display unit” of the present invention is the external monitor 16 will be described as an example.

The pressure measurement unit 20 includes a first pressure sensor 21, a second pressure sensor 22, and a third pressure sensor 23. The first pressure sensor 21 is provided in the circulation circuit 1R between the blood removing catheter 5 and the centrifugal pump 3. Specifically, the first pressure sensor 21 is provided on the blood removal tube 11. The first pressure sensor 21 detects a pressure value of blood flowing in the blood removal tube 11. The pressure value detected by the first pressure sensor 21 is an example of an “actual pressure measured by a first pressure sensor” of the present invention. The second pressure sensor 22 is provided in the circulation circuit 1R between the centrifugal pump 3 and the oxygenator 2. The second pressure sensor 22 detects a pressure value of blood flowing inside the circulation circuit 1R between the centrifugal pump 3 and the oxygenator 2. The pressure value detected by the second pressure sensor 22 is an example of an “actual pressure measured by a second pressure sensor” of the present invention. The third pressure sensor 23 is provided in the circulation circuit 1R between the oxygenator 2 and the blood feeding catheter 6. Specifically, the third pressure sensor 23 is provided on the blood feeding tube 12. The third pressure sensor 23 detects a pressure value of blood flowing in the blood feeding tube 12. The pressure value detected by the third pressure sensor 23 is an example of an “actual pressure measured by a third pressure sensor” of the present invention.

The flow rate measurement unit 24 is provided in the circulation circuit 1R between the blood removing catheter 5 and the oxygenator 2. Specifically, the flow rate measurement unit 24 is provided on the blood removal tube 11. Incidentally, the installation position of the flow rate measurement unit 24 is not limited to the blood removal tube 11, and may be any location in the circulation circuit 1R. The flow rate measurement unit 24 is, for example, a flow rate sensor, and detects a value of a flow rate of blood flowing inside the circulation circuit 1R. The value of the flow rate detected by the flow rate measurement unit 24 is an example of an “actual flow rate measured by a flow rate measurement unit” of the present invention.

The blood pressure measurement unit 25 is attached to the patient P and detects a pressure value (blood pressure value) of blood flowing through blood vessels of the patient P. The biological monitor 15 displays the blood pressure and other vital signs of patient P detected by the blood pressure measurement unit 25. The external monitor 16 displays the actual pressure measured by the pressure measurement unit 20 and the actual flow rate measured by the flow rate measurement unit 24 based on the control signal transmitted from the extracorporeal circulation management device 10. Details of an image displayed on the external monitor 16 will be described later.

FIG. 2 is a schematic diagram illustrating an extracorporeal circulation device according to a modification of the present embodiment. When a component of an extracorporeal circulation device 1A according to the modification illustrated in FIG. 2 is the same as the component of the extracorporeal circulation device 1 according to the present embodiment described with respect to FIG. 1, redundant descriptions will be omitted as appropriate, and differences will be mainly described hereinafter.

In the extracorporeal circulation device 1A according to the modification illustrated in FIG. 2, the first pressure sensor 21 is provided in the circulation circuit 1R between the centrifugal pump 3 and the oxygenator 2. The first pressure sensor 21 of the present modification detects a pressure value of blood flowing inside the circulation circuit 1R between the centrifugal pump 3 and the oxygenator 2. In this case, a pressure value of blood flowing inside the blood removal tube 11 is calculated by subtracting a head of the centrifugal pump 3 from the pressure value detected by the first pressure sensor 21.

The first pressure sensor 21 may be provided in the circulation circuit 1R between the blood removing catheter 5 and the centrifugal pump 3, specifically, in the blood removal tube 11, instead of between the centrifugal pump 3 and the oxygenator 2. In this case, the pressure value of blood flowing inside the circulation circuit 1R between the centrifugal pump 3 and the oxygenator 2 is calculated by adding the head of the centrifugal pump 3 to the pressure value detected by the first pressure sensor 21. In this manner, the first pressure sensor 21 is provided in the circulation circuit 1R at least between the blood removing catheter 5 and the centrifugal pump 3 or between the centrifugal pump 3 and the oxygenator 2 in the extracorporeal circulation device 1A according to the present modification.

The second pressure sensor 22 of the present modification is provided in the circulation circuit 1R between the oxygenator 2 and the blood feeding catheter 6. Specifically, the second pressure sensor 22 is provided on the blood feeding tube 12. The second pressure sensor 22 detects a pressure value of blood flowing in the blood feeding tube 12.

The pressure measurement unit 20 does not necessarily include the first pressure sensor 21, the second pressure sensor 22, and the third pressure sensor 23 as in the extracorporeal circulation device 1A according to the present modification, and can obtain an operation and an effect of the extracorporeal circulation device according to the present embodiment by providing the first pressure sensor 21 and the second pressure sensor 22. In the following description, the extracorporeal circulation device 1 described above with respect to FIG. 1 will be mainly taken as an example for convenience of the description, and the extracorporeal circulation device 1A illustrated in FIG. 2 will be taken as an example as necessary.

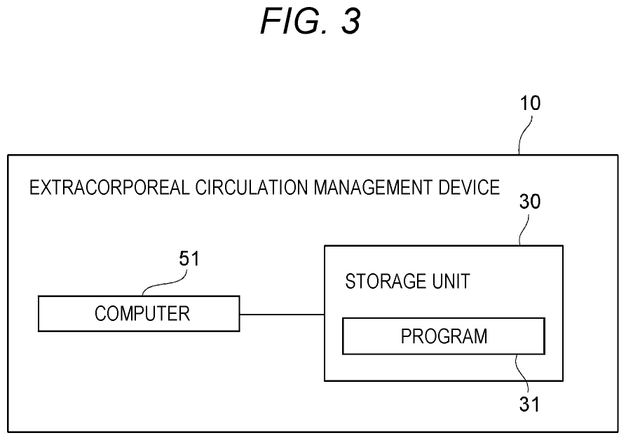

FIG. 3 is a block diagram illustrating a main configuration of an extracorporeal circulation management device according to the present embodiment. The extracorporeal circulation management device according to the present embodiment includes a computer 51 and a storage unit 30. The computer 51 includes a control unit 40 (see FIG. 4), reads a program 31 stored in the storage unit 30, and executes various calculations and processes. The storage unit 30 stores the program 31 (extracorporeal circulation management program) to be executed by the computer 51. The program 31 of the present embodiment is an example of the “extracorporeal circulation management program” of the present invention. Examples of the storage unit 30 include a hard disk drive (HDD) and the like. Incidentally, the program 31 is not limited to being stored in the storage unit 30, and may be distributed in a state of being stored in advance in a computer-readable storage medium or may be downloaded to the extracorporeal circulation management device 10 via a network. In addition, the storage unit 30 may be an external storage device connected to the computer 51.

Next, a main configuration of the extracorporeal circulation management device 10 according to the present embodiment will be further described with reference to the drawings. FIG. 4 is a block diagram illustrating the main configuration of the extracorporeal circulation device according to the present embodiment.

The extracorporeal circulation management device 10 according to the present embodiment includes the control unit 40, the storage unit 30, a touch panel 52, and a communication unit 53. The control unit 40 is, for example, a central processing unit (CPU) or the like, reads the program 31 (see FIG. 3) stored in the storage unit 30, and executes various calculations and processes. The control unit 40 includes a display processing unit 41, a notification processing unit 42, a standard pressure calculation unit 43, an actual pressure calculation unit 44, a differential flow rate calculation unit 45, a differential pressure calculation unit 46, and a standard flow rate calculation unit 47. The display processing unit 41, the notification processing unit 42, the standard pressure calculation unit 43, the actual pressure calculation unit 44, the differential flow rate calculation unit 45, the differential pressure calculation unit 46, and the standard flow rate calculation unit 47 are realized as the computer 51 executes the program 31 stored in the storage unit 30. Incidentally, the display processing unit 41, the notification processing unit 42, the standard pressure calculation unit 43, the actual pressure calculation unit 44, the differential flow rate calculation unit 45, the differential pressure calculation unit 46, and the standard flow rate calculation unit 47 may be realized by hardware, or may be realized by a combination of hardware and software. The storage unit 30 stores the program 31 described above with respect to FIG. 3, and includes an expected flow rate storage unit 32, a standard information storage unit 33, a pump characteristic storage unit 34, and a warning information storage unit 35.

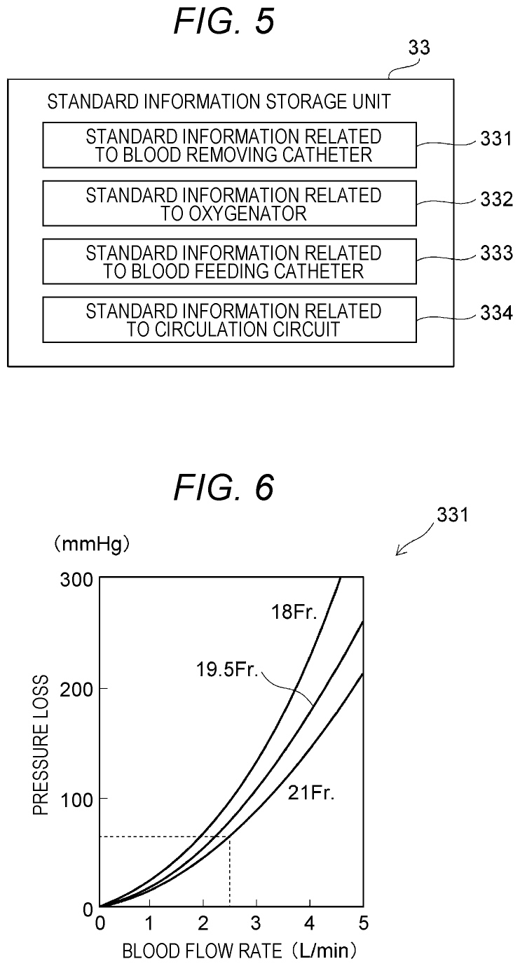

The display processing unit 41 executes a process of displaying an expected flow rate predicted in advance as an expected value of a flow rate of blood flowing inside the circulation circuit 1R and an actual flow rate, measured by the flow rate measurement unit 24 as an actually measured value of the flow rate of the blood flowing inside the circulation circuit, on the external monitor 16. In the present embodiment, the “expected flow rate” is a nominal or target flow rate value of blood calculated and determined by the standard flow rate calculation unit 47 using, for example, patient information (for example, height, weight, and the like) input by the touch panel 52 in response to the operator's operation on the touch panel 52 and standard information 334 (see FIG. 5) related to the circulation circuit 1R stored in the standard information storage unit 33. The expected flow rate calculated by the standard flow rate calculation unit 47 is transmitted from the standard flow rate calculation unit 47 to the expected flow rate storage unit 32 of the storage unit 30 and stored therein. Alternatively, the “expected flow rate” is a nominal flow rate value determined and predicted in advance by the operator or the like, and is an expected value of the flow rate of blood flowing inside the circulation circuit 1R. The expected flow rate determined by the operator or the like is input by, for example, the touch panel 52 and is stored in the expected flow rate storage unit 32 of the storage unit 30.

In addition, the display processing unit 41 executes a process of displaying both a standard pressure calculated based on the expected flow rate by referring to the standard information stored in the standard information storage unit 33 of the storage unit 30 and an actual pressure related to a device, calculated based on the actual pressure measured by the pressure measurement unit 20 as an actually measured value of the pressure of blood flowing inside the circulation circuit 1R and the actual flow rate measured by the flow rate measurement unit 24, on the external monitor 16. Details of the standard information and the standard pressure will be described later.

The notification processing unit 42 executes a process of providing notification of a warning when a predetermined condition is satisfied. For example, the warning notification is executed by displaying warning information (warning content) of the storage unit 30 on the external monitor 16. Alternatively, the warning notification may be executed, for example, by generation of light or sound.

The standard pressure calculation unit 43 refers to the standard information stored in the standard information storage unit 33 and calculates a standard pressure based on the expected flow rate stored in the expected flow rate storage unit 32. The standard information is information that represents a relation between the blood flow rate and a pressure loss occurring in each device such as the blood removing catheter 5, the oxygenator 2, and the blood feeding catheter 6 provided in the circulation circuit 1R. The standard pressure refers to the expected pressure loss occurring in each device provided in the circulation circuit 1R when the blood flow rate is the expected flow rate.

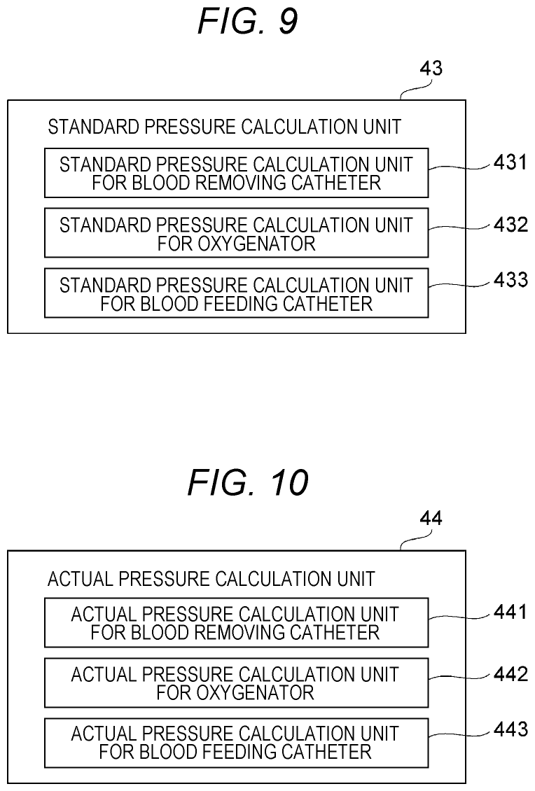

Here, the standard information storage unit 33 and the standard information stored in the standard information storage unit 33 will be further described with reference to FIGS. 5 to 8. In addition, the standard pressure calculation unit 43 will be further described with reference to FIG. 9.

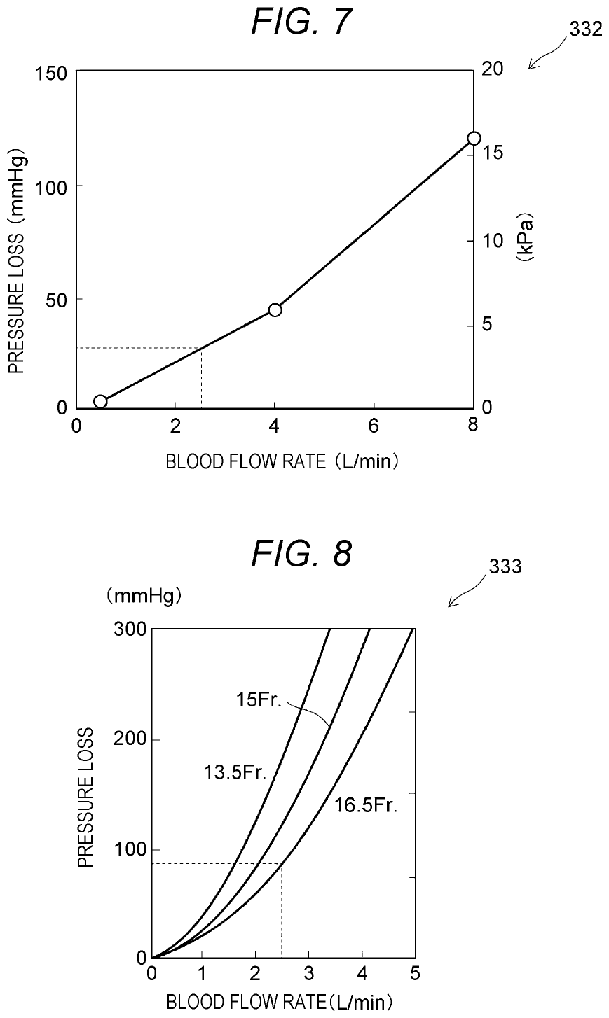

FIG. 5 is a schematic diagram illustrating the standard information storage unit of the present embodiment. FIG. 6 is a graph illustrating an example of standard information related to the blood removing catheter of the present embodiment. FIG. 7 is a graph illustrating an example of standard information related to the oxygenator of the present embodiment. FIG. 8 is a graph illustrating an example of standard information related to the blood feeding catheter of the present embodiment. FIG. 9 is a schematic diagram illustrating the standard pressure calculation unit of the present embodiment.

As illustrated in FIG. 5, the standard information stored in the standard information storage unit 33 includes standard information 331 related to the blood removing catheter 5, standard information 332 related to the oxygenator 2, standard information 333 related to the blood feeding catheter 6, and the standard information 334 related to the circulation circuit 1R.

An example of the standard information 331 related to the blood removing catheter 5 is given as illustrated in the graph illustrated in FIG. 6. That is, the horizontal axis of the graph illustrated in FIG. 6 is a flow rate (L/min) of blood flowing through the blood removing catheter 5. The vertical axis of the graph illustrated in FIG. 6 is a pressure loss (mmHg) that occurs in the blood removing catheter 5. As a condition of the graph illustrated in FIG. 6, the blood is bovine blood. Each of “18 Fr.”, “19.5 Fr.”, and “21 Fr.” described in the graph of FIG. 6 represents a French size of the blood removing catheter 5. For example, when the flow rate of blood flowing through the blood removing catheter 5 is 2.5 L/min in the case where the French size of the blood removing catheter 5 is “21 Fr.”, the pressure loss that occurs in the blood removing catheter 5 is about 63 mmHg.

An example of the standard information 332 related to oxygenator 2 is given as illustrated in the graph illustrated in FIG. 7. That is, the horizontal axis of the graph illustrated in FIG. 7 is a flow rate (L/min) of blood flowing through the oxygenator 2. The vertical axis of the graph illustrated in FIG. 7 is a pressure loss (mmHg) that occurs in the oxygenator 2. As a condition of the graph illustrated in FIG. 7, the blood is bovine blood. For example, when the flow rate of blood flowing through the oxygenator 2 is 2. 5 L/min, the pressure loss generated in the oxygenator 2 is about 28 mmHg.

An example of the standard information 333 related to the blood feeding catheter 6 is given as illustrated in the graph illustrated in FIG. 8. That is, the horizontal axis of the graph illustrated in FIG. 8 is a flow rate (L/min) of blood flowing through the blood feeding catheter 6. The vertical axis of the graph illustrated in FIG. 8 is a pressure loss (mmHg) that occurs in the blood feeding catheter 6. As a condition of the graph illustrated in FIG. 8, the blood is bovine blood. Each of “13.5 Fr.”, “15 Fr.” and “16.5 Fr.” described in the graph of FIG. 8 represents a French size of the blood feeding catheter 6. For example, when the flow rate of blood flowing through the blood feeding catheter 6 is 2.5 L/min in the case where the French size of the blood feeding catheter 6 is “16.5 Fr.”, the pressure loss that occurs in the blood feeding catheter 6 is about 87 mmHg.

The standard information 334 related to the circulation circuit 1R includes the standard information 331 related to the blood removing catheter 5, the standard information 332 related to the oxygenator 2, the standard information 333 related to the blood feeding catheter 6, and standard information related to tubes used in the circulation circuit 1R. Examples of the standard information related to the tubes used in the circulation circuit 1R can include standard information related to the blood removal tube 11, standard information related to the blood feeding tube 12, and the like.

As illustrated in FIG. 9, the standard pressure calculation unit 43 of the present embodiment includes a standard pressure calculation unit 431 related to the blood removing catheter 5, a standard pressure calculation unit 432 related to the oxygenator 2, and a standard pressure calculation unit 433 related to the blood feeding catheter 6. The standard pressure calculation unit 431 related to the blood removing catheter 5 refers to the standard information 331 related to the blood removing catheter 5 and calculates a standard pressure related to the blood removing catheter 5 based on the expected flow rate stored in the expected flow rate storage unit 32. The standard pressure related to the blood removing catheter 5 refers to a pressure loss that occurs in the blood removing catheter 5 when the blood flow rate is the expected flow rate. For example, when the expected flow rate stored in the expected flow rate storage unit 32 is 2.5 L/min, the standard pressure calculation unit 431 related to the blood removing catheter 5 calculates about 63 mmHg as the standard pressure (i.e., pressure loss or drop) related to the blood removing catheter 5.

The standard pressure calculation unit 432 related to the oxygenator 2 refers to the standard information 332 related to the oxygenator 2 and calculates a standard pressure related to the oxygenator 2 based on the expected flow rate stored in the expected flow rate storage unit 32. The standard pressure related to the oxygenator 2 refers to a pressure loss that occurs in the oxygenator 2 when the blood flow rate is the expected flow rate. For example, when the expected flow rate stored in the expected flow rate storage unit 32 is 2.5 L/min, the standard pressure calculation unit 432 related to the oxygenator 2 calculates about 28 mmHg as the standard pressure (loss) related to the oxygenator 2.

The standard pressure calculation unit 433 related to the blood feeding catheter 6 refers to the standard information 333 related to the blood feeding catheter 6, and calculates a standard pressure related to the blood feeding catheter 6 based on the expected flow rate stored in the expected flow rate storage unit 32. The standard pressure related to the blood feeding catheter 6 refers to a pressure loss that occurs in the blood feeding catheter 6 when the blood flow rate is the expected flow rate. For example, when the expected flow rate stored in the expected flow rate storage unit 32 is 2.5 L/min, the standard pressure calculation unit 433 related to the blood feeding catheter 6 calculates about 87 mmHg as the standard pressure (loss) related to the blood feeding catheter 6.

Subsequently, the main configuration of the extracorporeal circulation management device 10 will be further described returning to FIG. 4. The actual pressure calculation unit 44 calculates a pressure loss that actually occurs in each device, such as the blood removing catheter 5, the oxygenator 2, and the blood feeding catheter 6 provided in the circulation circuit 1R, based on the actual pressure measured by the pressure measurement unit 20 and the actual flow rate measured by the flow rate measurement unit 24.

Here, the actual pressure calculation unit 44 will be further described with reference to FIG. 10. FIG. 10 is a schematic diagram illustrating the actual pressure calculation unit of the present embodiment. As illustrated in FIG. 10, the actual pressure calculation unit 44 of the present embodiment includes an actual pressure calculation unit 441 related to the blood removing catheter 5, an actual pressure calculation unit 442 related to the oxygenator 2, and an actual pressure calculation unit 443 related to the blood feeding catheter 6.

The actual pressure calculation unit 441 related to the blood removing catheter 5 calculates an actual pressure related to the blood removing catheter 5 based on the actual pressure measured by the first pressure sensor 21 and the actual flow rate measured by the flow rate measurement unit 24. Specifically, the actual pressure calculation unit 441 related to the blood removing catheter 5 calculates a value, obtained by subtracting a blood pressure (for example, central venous pressure (CVP)) displayed on the biological monitor 15 from an actually measured value measured by the first pressure sensor 21, as a pressure loss that actually occurs in the blood removing catheter 5.

Incidentally, in the extracorporeal circulation device 1A according to the modification described above with respect to FIG. 2, the actual pressure calculation unit 441 related to the blood removing catheter 5 calculates the actual pressure related to the blood removing catheter 5 based on the actual pressure measured by the first pressure sensor 21, the head of the centrifugal pump 3, and the actual flow rate measured by the flow rate measurement unit 24. Specifically, the actual pressure calculation unit 441 related to the blood removing catheter 5 calculates a value, obtained by subtracting the head of the centrifugal pump 3 from the actually measured value measured by the first pressure sensor 21, as a pressure value of blood flowing inside the blood removal tube 11 (blood pressure inside the blood removal tube 11). Further, the actual pressure calculation unit 441 related to the blood removing catheter 5 calculates a value, obtained by subtracting a blood pressure displayed on the biological monitor 15 (blood pressure of the patient P detected by the blood pressure measurement unit 25: for example, central venous pressure (CVP)) from the blood pressure in the blood removal tube 11, as a pressure loss that actually occurs in the blood removing catheter 5.

The actual pressure calculation unit 442 related to the oxygenator 2 calculates an actual pressure related to the oxygenator 2 based on the actual pressure measured by the second pressure sensor 22, the actual pressure measured by the third pressure sensor 23, and the actual flow rate measured by the flow rate measurement unit 24. Specifically, the actual pressure calculation unit 442 related to the oxygenator 2 calculates a value, obtained by subtracting an actually measured value measured by the second pressure sensor 22 from an actually measured value measured by the third pressure sensor 23, as a pressure loss that actually occurs in the oxygenator 2.

Incidentally, in the extracorporeal circulation device 1A according to the modification described above with respect to FIG. 2, the actual pressure calculation unit 442 related to the oxygenator 2 calculates the actual pressure related to the oxygenator 2 based on the actual pressure measured by the first pressure sensor 21, the actual pressure measured by the second pressure sensor 22, and the actual flow rate measured by the flow rate measurement unit 24. Specifically, the actual pressure calculation unit 442 related to the oxygenator 2 calculates a value, obtained by subtracting the actually measured value measured by the first pressure sensor 21 from the actually measured value measured by the second pressure sensor 22, as a pressure loss that actually occurs in the oxygenator 2. Alternatively, in the extracorporeal circulation device 1A according to the modification described with respect to FIG. 2, the actual pressure calculation unit 442 related to the oxygenator 2 calculates a value, obtained by adding the head of the centrifugal pump 3 to the actually measured value measured by the first pressure sensor 21, as a pressure value flowing in a tube between the centrifugal pump 3 and the oxygenator 2 (a blood pressure in the tube between the centrifugal pump 3 and the oxygenator 2) when the first pressure sensor 21 is provided in the circulation circuit 1R between the blood removing catheter 5 and the centrifugal pump 3. Further, the actual pressure calculation unit 442 related to the oxygenator 2 calculates a value, obtained by subtracting the blood pressure in the tube between the centrifugal pump 3 and the oxygenator 2 from the actually measured value measured by the second pressure sensor 22, as the pressure loss that actually occurs in the oxygenator 2.

The actual pressure calculation unit 443 related to the blood feeding catheter 6 calculates an actual pressure related to the blood feeding catheter 6 based on the actual pressure measured by the third pressure sensor 23 and the actual flow rate measured by the flow rate measurement unit 24. Specifically, the actual pressure calculation unit 443 related to the blood feeding catheter 6 calculates a value, obtained by subtracting the actually measured value measured by the third pressure sensor 23 from a blood pressure (for example, average blood pressure) displayed on the biological monitor 15, as a pressure loss that actually occurs in the blood feeding catheter 6.

Incidentally, in the extracorporeal circulation device 1A according to the modification described with respect to FIG. 2, the actual pressure calculation unit 443 related to the blood feeding catheter 6 calculates the actual pressure related to the blood feeding catheter 6 based on the actual pressure measured by the second pressure sensor 22 and the actual flow rate measured by the flow rate measurement unit 24. Specifically, the actual pressure calculation unit 443 related to the blood feeding catheter 6 calculates a value, obtained by subtracting the actually measured value measured by the second pressure sensor 22 from the blood pressure (for example, average blood pressure) displayed on the biological monitor 15, as the pressure loss that actually occurs in the blood feeding catheter 6.

Subsequently, the main configuration of the extracorporeal circulation management device 10 will be further described returning to FIG. 4. The differential flow rate calculation unit 45 calculates a differential flow rate representing a difference between an expected flow rate and an actual flow rate. Specifically, the differential flow rate calculation unit 45 calculates a difference between the expected flow rate stored in the expected flow rate storage unit 32 and the actually measured value (actual flow rate) measured by the flow rate measurement unit 24 as the differential flow rate.

The differential pressure calculation unit 46 calculates a differential pressure representing a difference between a standard pressure and an actual pressure. Specifically, the differential pressure calculation unit 46 calculates a difference between the standard pressure calculated by the standard pressure calculation unit 43 and the actual pressure related to each device calculated by the actual pressure calculation unit 44 as the differential pressure.

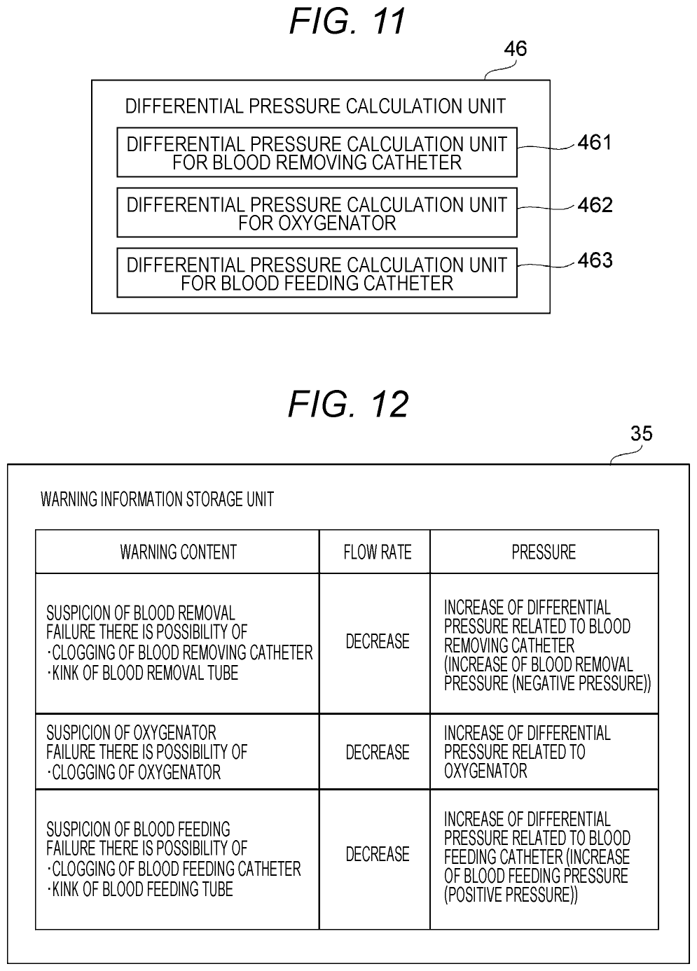

Here, the differential pressure calculation unit 46 will be further described with reference to FIG. 11. FIG. 11 is a schematic diagram illustrating the differential pressure calculation unit of the present embodiment. As illustrated in FIG. 11, the differential pressure calculation unit 46 of the present embodiment includes a differential pressure calculation unit 461 related to the blood removing catheter 5, a differential pressure calculation unit 462 related to the oxygenator 2, and a differential pressure calculation unit 463 related to the blood feeding catheter 6.

The differential pressure calculation unit 461 related to the blood removing catheter 5 calculates a differential pressure related to the blood removing catheter 5 which represents a difference between the standard pressure related to the blood removing catheter 5 and the actual pressure related to the blood removing catheter 5. Specifically, the differential pressure calculation unit 461 related to the blood removing catheter 5 calculates a difference between the standard pressure related to the blood removing catheter 5 calculated by the standard pressure calculation unit 431 related to the blood removing catheter 5 and the actual pressure related to the blood removing catheter 5 calculated by the actual pressure calculation unit 441 related to the blood removing catheter 5 as the differential pressure related to the blood removing catheter 5.

The differential pressure calculation unit 462 related to the oxygenator 2 calculates a differential pressure related to the oxygenator 2 which represents a difference between the standard pressure related to the oxygenator 2 and the actual pressure related to the oxygenator 2. Specifically, the differential pressure calculation unit 462 related to the oxygenator 2 calculates a difference between the standard pressure related to the oxygenator 2 calculated by the standard pressure calculation unit 432 related to the oxygenator 2 and the actual pressure related to the oxygenator 2 calculated by the actual pressure calculation unit 442 related to the oxygenator 2 as the differential pressure related to the oxygenator 2.

The differential pressure calculation unit 463 related to the blood feeding catheter 6 calculates a differential pressure related to the blood feeding catheter 6 which represents a difference between the standard pressure related to the blood feeding catheter 6 and the actual pressure related to the blood feeding catheter 6. Specifically, the differential pressure calculation unit 463 related to the blood feeding catheter 6 calculates a difference between the standard pressure related to the blood feeding catheter 6 calculated by the standard pressure calculation unit 433 related to the blood feeding catheter 6 and the actual pressure related to the blood feeding catheter 6 calculated by the actual pressure calculation unit 443 related to the blood feeding catheter 6 as the differential pressure related to the blood feeding catheter 6.

Subsequently, the main configuration of the extracorporeal circulation management device 10 will be further described returning to FIG. 4. The expected flow rate storage unit 32 stores the expected flow rate calculated by the standard flow rate calculation unit 47 and transmitted from the standard flow rate calculation unit 47. Alternatively, the expected flow rate storage unit 32 stores the expected flow rate determined by the operator or the like and input by, for example, the touch panel 52. The standard information storage unit 33 is given as described above with respect to FIGS. 5 to 8.

The pump characteristic storage unit stores information on a characteristic of the centrifugal pump 3. Examples of the information on the characteristic of the centrifugal pump 3 include a graph illustrating a relation between a flow rate (L/min) of blood sent by the centrifugal pump 3 and a head (mmHg) of the centrifugal pump 3. For example, in the graph illustrating the characteristic of the centrifugal pump 3, the relation between the flow rate (L/min) of blood sent by the centrifugal pump 3 and the head (mmHg) of the centrifugal pump 3 is set depending on a rotational speed (rpm) of the centrifugal pump 3.

The warning information storage unit 35 stores warning information (warning content) notification of which is provided by the notification processing unit 42. Here, the differential pressure calculation unit 46 will be further described with reference to FIG. 12. FIG. 12 is a schematic diagram illustrating the warning information storage unit of the present embodiment.

As illustrated in FIG. 12, the warning information storage unit 35 stores warning information data that can be expected by combining a flow rate and a pressure. For example, in a case where the actual flow rate measured by the flow rate measurement unit 24 decreases and the differential pressure related to the blood removing catheter 5 increases, the notification processing unit 42 refers to the warning information stored in the warning information storage unit 35 and displays that there is a suspicion of blood removal failure on the external monitor 16. For example, the notification processing unit 42 displays that there is a “possibility of clogging of the blood removing catheter 5” due to the distal end of the blood removing catheter 5 coming into contact with a blood vessel wall or there is a “possibility of a kink of the blood removal tube 11” on the external monitor 16 as the suspicion of the blood removal failure. Incidentally, the case where the differential pressure related to the blood removing catheter 5 increases corresponds to, for example, a case where a blood removal pressure (negative pressure) increases.

In addition, for example, in a case where the actual flow rate measured by the flow rate measurement unit 24 decreases and the differential pressure related to the oxygenator 2 increases, the notification processing unit 42 refers to the warning information stored in the warning information storage unit 35 and displays that there is a suspicion of oxygenator failure on the external monitor 16. For example, the notification processing unit 42 displays that there is a “possibility of clogging of the oxygenator 2” due to the life of the oxygenator 2 or the like on the external monitor 16 as the suspicion of oxygenator failure.

In addition, for example, in a case where the actual flow rate measured by the flow rate measurement unit 24 decreases and the differential pressure related to the blood feeding catheter 6 increases, the notification processing unit 42 refers to the warning information stored in the warning information storage unit 35 and displays that there is a suspicion of blood feeding failure on the external monitor 16. For example, the notification processing unit 42 displays that there is a “possibility of clogging of the blood feeding catheter 6” due to a distal end of the blood feeding catheter 6 coming into contact with a blood vessel wall or there is a “possibility of a kink of the blood feeding tube 12” on the external monitor 16 as the suspicion of the blood feeding failure. Incidentally, the case where the differential pressure of the blood feeding catheter 6 increases corresponds to, for example, a case where a blood feeding pressure (positive pressure) increases.

Subsequently, the main configuration of the extracorporeal circulation management device 10 will be further described returning to FIG. 4. The communication unit 53 communicates with the drive motor 4, the biological monitor 15, the external monitor 16, the pressure measurement unit 20, and the flow rate measurement unit 24, and transmits and receives various types of information and various signals.

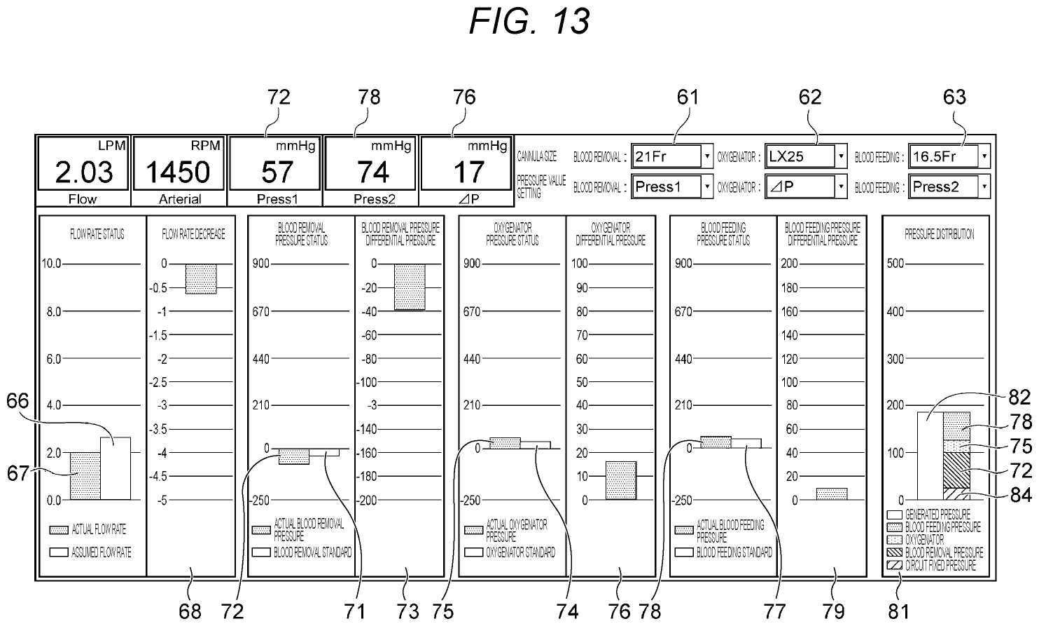

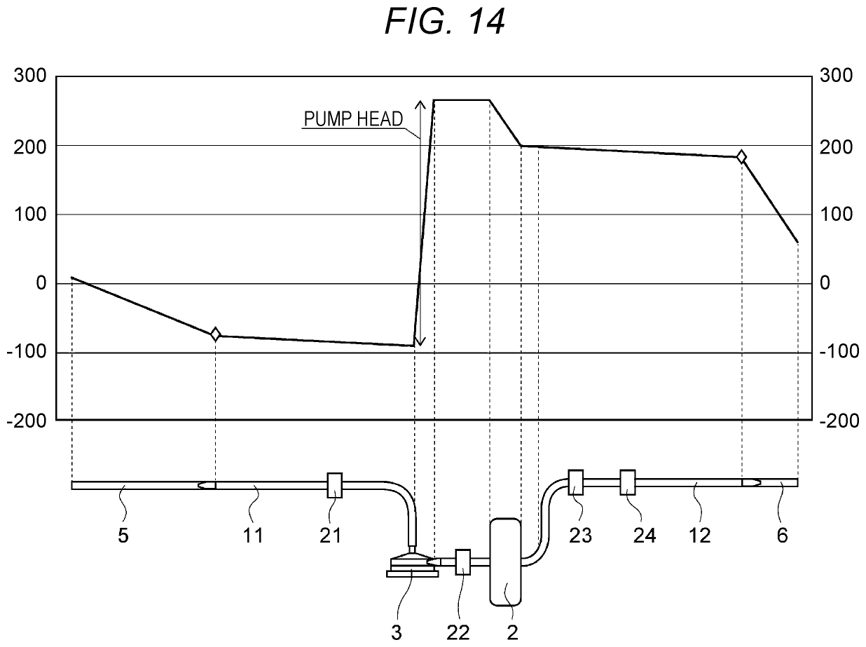

Next, an example of an image displayed on the external monitor 16 of the present embodiment will be described with reference to the drawings. FIG. 13 is a schematic diagram illustrating an example of the image displayed on the external monitor according to the present embodiment. FIG. 14 is a schematic diagram illustrating an example of a relation between arrangements of the circulation circuit and each device of the extracorporeal circulation device according to the present embodiment and a pressure loss.

As illustrated in FIG. 13, the display processing unit 41 of the extracorporeal circulation management device 10 according to the present embodiment displays an expected flow rate 66 (labelled in FIG. 13 as “Assumed Flow Rate”) stored in the expected flow rate storage unit 32 and an actual flow rate 67 measured by the flow rate measurement unit 24 on the external monitor 16, using side-by-side bar graphs. In addition, the display processing unit 41 displays standard pressures 71, 74, and 77 calculated by the standard pressure calculation unit 43 and actual pressures 72, 75, and 78 related to the respective devices calculated by the actual pressure calculation unit 44 on the external monitor 16.

Further, the display processing unit 41 displays a differential flow rate 68, which represents a difference between the expected flow rate 66 and the actual flow rate 67, and differential pressures 73, 76, and 79 each of which represents a difference between each of the standard pressures 71, 74, and 77 and each of the actual pressures 72, 75, and 78 related to the respective devices on the external monitor 16.

To be specific, the display processing unit 41 displays the standard pressure 71 related to the blood removing catheter 5, calculated based on the expected flow rate 66 by the standard pressure calculation unit 431 related to the blood removing catheter 5, and the actual pressure 72 related to the blood removing catheter 5, calculated by the actual pressure calculation unit 441 related to the blood removing catheter 5 based on the actual pressure measured by the first pressure sensor 21 and the actual flow rate 67 measured by the flow rate measurement unit 24, on the external monitor 16. In the example of the image illustrated in FIG. 13, the display processing unit 41 displays the standard pressure 71 related to the blood removing catheter 5 and the actual pressure 72 related to the blood removing catheter 5 in parallel. Further, the display processing unit 41 displays the differential pressure 73 related to the blood removing catheter 5 calculated by the differential pressure calculation unit 461 related to the blood removing catheter 5, which represents the difference between the standard pressure 71 related to the blood removing catheter 5 and the actual pressure 72 related to the blood removing catheter 5, on the external monitor 16. In the example of the image illustrated in FIG. 13, the display processing unit 41 displays the standard pressure 71 related to the blood removing catheter 5, the actual pressure 72 related to the blood removing catheter 5, and the differential pressure 73 related to the blood removing catheter 5 in parallel.

In addition, the display processing unit 41 displays the standard pressure 74 related to the oxygenator 2, calculated based on the expected flow rate 66 by the standard pressure calculation unit 432 related to the oxygenator 2, and the actual pressure 75 related to the oxygenator 2, calculated by the actual pressure calculation unit 442 related to the oxygenator 2 based on the actual pressures measured by the second pressure sensor 22 and the third pressure sensor 23 and the actual flow rate 67 measured by the flow rate measurement unit 24, on the external monitor 16. In the example of the image illustrated in FIG. 13, the display processing unit 41 displays the standard pressure 74 related to the oxygenator 2 and the actual pressure 75 related to the oxygenator 2 in parallel. Further, the display processing unit 41 displays the differential pressure 76 related to the oxygenator 2 calculated by the differential pressure calculation unit 462 related to the oxygenator 2, which represents the difference between the standard pressure 74 related to the oxygenator 2 and the actual pressure 75 related to the oxygenator 2, on the external monitor 16. In the example of the image illustrated in FIG. 13, the display processing unit 41 displays the standard pressure 74 related to the oxygenator 2, the actual pressure 75 related to the oxygenator 2, and the differential pressure 76 related to the oxygenator 2 in parallel.

In addition, the display processing unit 41 displays the standard pressure 77 related to the blood feeding catheter 6, calculated based on the expected flow rate 66 by the standard pressure calculation unit 433 related to the blood feeding catheter 6, and the actual pressure 78 related to the blood feeding catheter 6, calculated by the actual pressure calculation unit 443 related to the blood feeding catheter 6 based on the actual pressure measured by the third pressure sensor 23 and the actual flow rate 67 measured by the flow rate measurement unit 24, on the external monitor 16. In the example of the image illustrated in FIG. 13, the display processing unit 41 displays the standard pressure 77 related to the blood feeding catheter 6 and the actual pressure 78 related to the blood feeding catheter 6 in parallel. Further, the display processing unit 41 displays the differential pressure 79 related to the blood feeding catheter 6 calculated by the differential pressure calculation unit 463 related to the blood feeding catheter 6, which represents the difference between the standard pressure 77 related to the blood feeding catheter 6 and the actual pressure 78 related to the blood feeding catheter 6, on the external monitor 16. In the example of the image illustrated in FIG. 13, the display processing unit 41 displays the standard pressure 77 related to the blood feeding catheter 6, the actual pressure 78 related to the blood feeding catheter 6, and the differential pressure 79 related to the blood feeding catheter 6 in parallel.

According to the extracorporeal circulation device 1 and the extracorporeal circulation management device 10 of the present embodiment, the operator or the like can easily grasp a discrepancy or deviation between the expected flow rate 66 of blood in the circulation circuit 1R and the actual flow rate 67 of the blood actually flowing in the circulation circuit 1R by confirming the external monitor 16. In addition, the operator or the like can easily grasp a discrepancy or deviation between each of the standard pressures 71, 74, and 77 calculated based on the expected flow rate 66 and each of the actual pressures 72, 75, and 78 related to the respective devices (the blood removing catheter 5, the oxygenator 2, and the blood feeding catheter 6) by observing the external monitor 16. Therefore, when there is the discrepancy or deviation between the expected flow rate 66 and the actual flow rate 67, the operator or the like can easily grasp the occurrence of the discrepancy or deviation between the expected flow rate 66 and the actual flow rate 67 and grasp a location of the circulation circuit 1R where the discrepancy or deviation between the standard pressure 71, 74, or 77 and the actual pressure 72, 75, or 78 has occurred and easily grasp that a cause of circulation failure exists near the location by confirming the external monitor 16. That is, the location of the circulation circuit 1R where the discrepancy or deviation between the standard pressure 71, 74, or 77 and the actual pressure 72, 75, or 78 has occurred corresponds to a portion where the pressure measurement unit 20 is provided or a portion of each device provided near the pressure measurement unit 20. In this manner, the operator or the like can easily grasp the cause of the circulation failure in the extracorporeal circulation.

In addition, the differential flow rate 68 and the differential pressures 73, 76, and 79 are further displayed on the external monitor 16 as described above. As a result, the operator or the like can more easily grasp the discrepancy or deviation between the expected flow rate 66 and the actual flow rate 67 and the discrepancy or deviation between the standard pressure 71, 74, or 77 and the actual pressure 72, 75, or 78 by confirming the external monitor 16. As described above, the standard pressure 71, 74, or 77 is calculated based on the standard information 331, 332, or 333 stored in the standard information storage unit 33 and the expected flow rate 66 stored in the expected flow rate storage unit 32. That is, the standard pressures 71, 74, and 77 change depending on the expected flow rate 66. Therefore, a discrepancy or deviation sometimes occurs between the standard pressure 71, 74, or 77 and the actual pressure 72, 75, or 78 even if the actual pressure 72, 75, or 78 does not change at first glance. On the other hand, according to the extracorporeal circulation device 1 and the extracorporeal circulation management device 10 of the present embodiment, the operator or the like can more easily grasp the discrepancy or deviation between the standard pressure 71, 74, or 77 and the actual pressure 72, 75, or 78 by confirming the external monitor 16.

In addition, as illustrated in FIG. 13, the standard pressure 71, the actual pressure 72, and the differential pressure 73 related to the blood removing catheter 5, the standard pressure 74, the actual pressure 75, and the differential pressure 76 related to the oxygenator 2, and the standard pressure 77, the actual pressure 78, and the differential pressure 79 related to the blood feeding catheter 6 are displayed in parallel according to the actual arrangement of each device or per actual arrangement of each device. As a result, the operator or the like can more easily grasp the discrepancy or deviation between the standard pressure 71, 74, or 77 and the actual pressure 72, 75, or 78, and more concretely and easily grasp a location of the circulation circuit 1R where the discrepancy or deviation between the standard pressure 71, 74, or 77 and the actual pressure 72, 75, or 78 has occurred and easily grasp that a cause of circulation failure exists near the location by observing the external monitor 16 as one display unit.

In addition, as illustrated in FIG. 13, all the expected flow rate 66, the actual flow rate 67, the differential flow rate 68, the standard pressure 71 related to the blood removing catheter 5, the standard pressure 74 related to the oxygenator 2, the standard pressure 77 related to the blood feeding catheter 6, the actual pressure 72 related to the blood removing catheter 5, the actual pressure 75 related to the oxygenator 2, the actual pressure 78 related to the blood feeding catheter 6, the differential pressure 73 related to the blood removing catheter 5, the differential pressure 76 related to the oxygenator 2, and the differential pressure 79 related to the blood feeding catheter 6 are displayed simultaneously and in parallel on the external monitor 16 as one display unit. As a result, the operator or the like can more easily grasp the discrepancy or deviation between the standard pressure 71, 74, or 77 and the actual pressure 72, 75, or 78, and more concretely and easily grasp a location of the circulation circuit 1R where the discrepancy or deviation between the standard pressure 71, 74, or 77 and the actual pressure 72, 75, or 78 has occurred and easily grasp that a cause of circulation failure exists near the location by confirming the external monitor 16 as one display unit. That is, a location of the circulation circuit 1R where a discrepancy or deviation between the standard pressure 71, 74, or 77 and the actual pressure 72, 75, or 78 has occurred corresponds to a portion where a pressure sensor detecting an abnormal value among the first pressure sensor 21, the second pressure sensor 22, and the third pressure sensor 23 is provided or a portion of an instrument element provided near the pressure sensor detecting the abnormal value among the blood removing catheter 5, the oxygenator 2, and the blood feeding catheter 6. In addition, the image (bar graph) displayed on the external monitor 16 changes in real time. As a result, the operator or the like can immediately grasp the discrepancy or deviation between the standard pressure 71, 74, or 77 and the actual pressure 72, 75, or 78.

In addition, as illustrated in FIG. 13, the display processing unit 41 displays a French size 61 of the blood removing catheter 5, a type 62 of the oxygenator 2, and a French size 63 of the blood feeding catheter 6 on the external monitor 16. The operator or the like can easily change settings of at least any of the French size 61 of the blood removing catheter 5, the type 62 of the oxygenator 2, and the French size 63 of the blood feeding catheter 6 on the image displayed on the external monitor 16. As a result, the operator or the like can change settings of the standard pressure 71 related to the blood removing catheter 5, the standard pressure 74 related to the oxygenator 2, and the standard pressure 77 related to the blood feeding catheter 6 by changing settings of at least any of the French size 61 of the blood removing catheter 5, the type 62 of the oxygenator 2, and the French size 63 of the blood feeding catheter 6 on the image displayed on the external monitor 16. A relation between the French size 61 of the blood removing catheter 5 and the standard pressure 71 related to the blood removing catheter 5 and a relation between the French size 63 of the blood feeding catheter 6 and the standard pressure 77 related to the blood feeding catheter 6 are the same as those described above with respect to FIGS. 6 and 8.

Further, the display processing unit 41 displays an overall pressure distribution 81 of the extracorporeal circulation device 1 on the external monitor 16. The overall pressure distribution 81 of the extracorporeal circulation device 1 includes a head 82 of the centrifugal pump 3, the actual pressure 72 related to the blood removing catheter 5, the actual pressure 75 related to the oxygenator 2, the actual pressure 78 related to the blood feeding catheter 6, and a circuit fixed pressure 84. The circuit fixed pressure 84 is a pressure applied to a tube connecting the blood removing catheter 5, the centrifugal pump 3, the oxygenator 2, and the blood feeding catheter 6, and is, for example, a pressure applied to the blood removal tube 11, a pressure applied to the blood feeding tube 12, or the like.

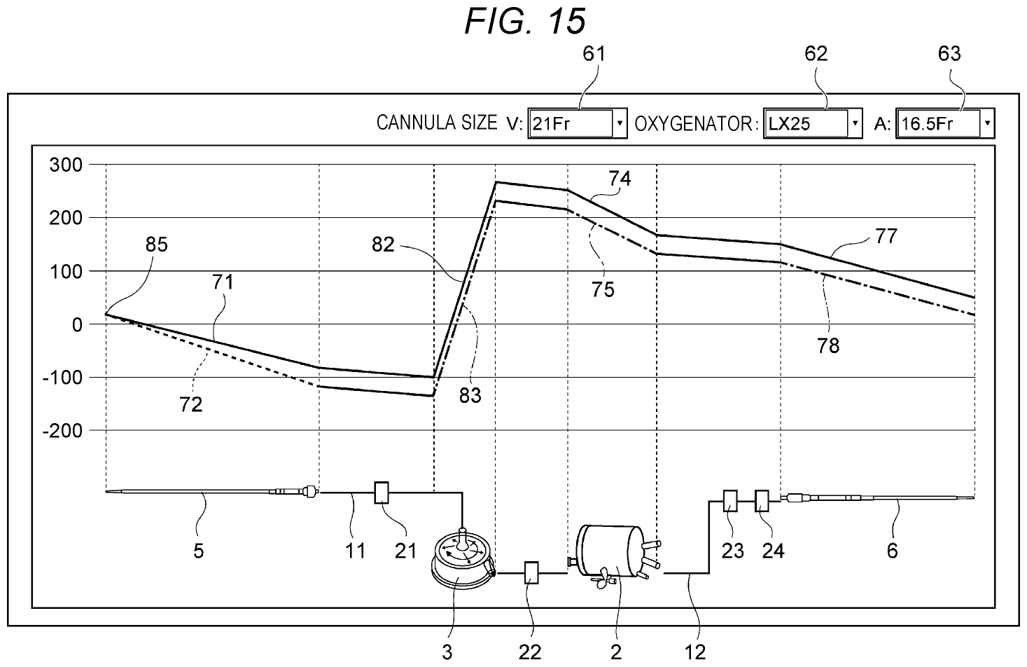

FIG. 15 is a schematic diagram illustrating another example of the image displayed on the external monitor of the present embodiment. On the image displayed on the external monitor 16 described above with respect to FIG. 13, the standard pressure 71 related to the blood removing catheter 5, the standard pressure 74 related to the oxygenator 2, the standard pressure 77 related to the blood feeding catheter 6, the actual pressure 72 related to the blood removing catheter 5, the actual pressure 75 related to the oxygenator 2, the actual pressure 78 related to the blood feeding catheter 6, the differential pressure 73 related to the blood removing catheter 5, the differential pressure 76 related to the oxygenator 2, the differential pressure 79 related to the blood feeding catheter 6, and the head 82 of the centrifugal pump 3 are illustrated using bar graphs. In contrast, on the image illustrated in FIG. 15, blood pressure information displayed on the biological monitor 15, such as the standard pressure 71 related to the blood removing catheter 5, the standard pressure 74 related to the oxygenator 2, the standard pressure 77 related to the blood feeding catheter 6, the actual pressure 72 related to the blood removing catheter 5, the actual pressure 75 related to the oxygenator 2, the actual pressure 78 related to the blood feeding catheter 6, the differential pressure 73 related to the blood removing catheter 5, the differential pressure 76 related to the oxygenator 2, and the differential pressure 79 related to the blood feeding catheter 6, the standard head 82 of the centrifugal pump 3, an actual head 83 of the centrifugal pump 3, and a central venous pressure (CVP) 85, is illustrated using a line graph. In this regard, the image illustrated in FIG. 15 is different from the image illustrated in FIG. 13.

On the image illustrated in FIG. 15, the operator or the like can more easily grasp a discrepancy or deviation between the standard pressure 71, 74, or 77 and the actual pressure 72, 75, or 78 while confirming the relation between the arrangements of the circulation circuit 1R and the respective devices of the extracorporeal circulation device 1 and the pressure loss using the external monitor 16. In other words, the operator or the like can more concretely and easily grasp a location of the circulation circuit 1R where the discrepancy or deviation between the standard pressure 71, 74, or 77 and the actual pressure 72, 75, or 78 has occurred, and easily grasp that a cause of circulation failure exists near the location. In addition, the image (line graph) displayed on the external monitor 16 changes in real time. As a result, the operator or the like can immediately grasp the discrepancy or deviation between the standard pressure 71, 74, or 77 and the actual pressure 72, 75, or 78.

In the example of the image illustrated in FIG. 15, it can be seen that the discrepancy or deviation occurs between the standard pressure 71 related to the blood removing catheter 5 and the actual pressure 72 related to the blood removing catheter 5. On the other hand, it can be seen that there is no discrepancy or deviation between the standard pressure 74 related to the oxygenator 2 and the actual pressure 75 related to the oxygenator 2, between the standard pressure 77 related to the blood feeding catheter 6 and the actual pressure 78 related to the blood feeding catheter 6, and between the standard head 82 of the centrifugal pump 3 and the actual head 83 of the centrifugal pump 3. As a result, in the example of the image illustrated in FIG. 15, the operator or the like can grasp that there is a “possibility of clogging of the blood removing catheter 5” due to the distal end of the blood removing catheter 5 coming into contact with the blood vessel wall or there is a “possibility of a kink of the blood removal tube 11” as a suspicion of blood removal failure.

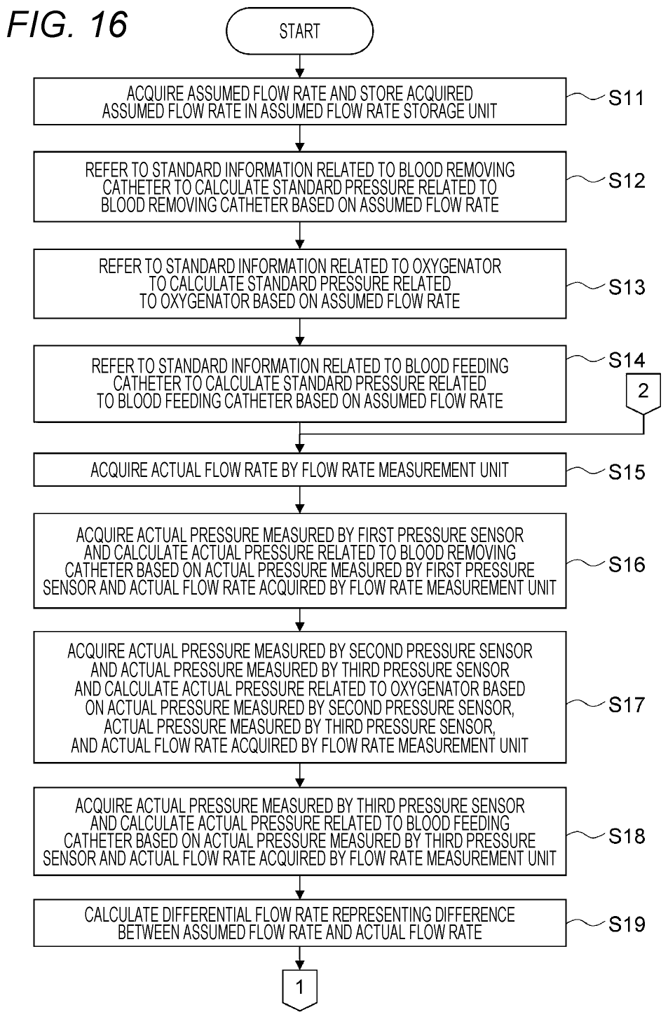

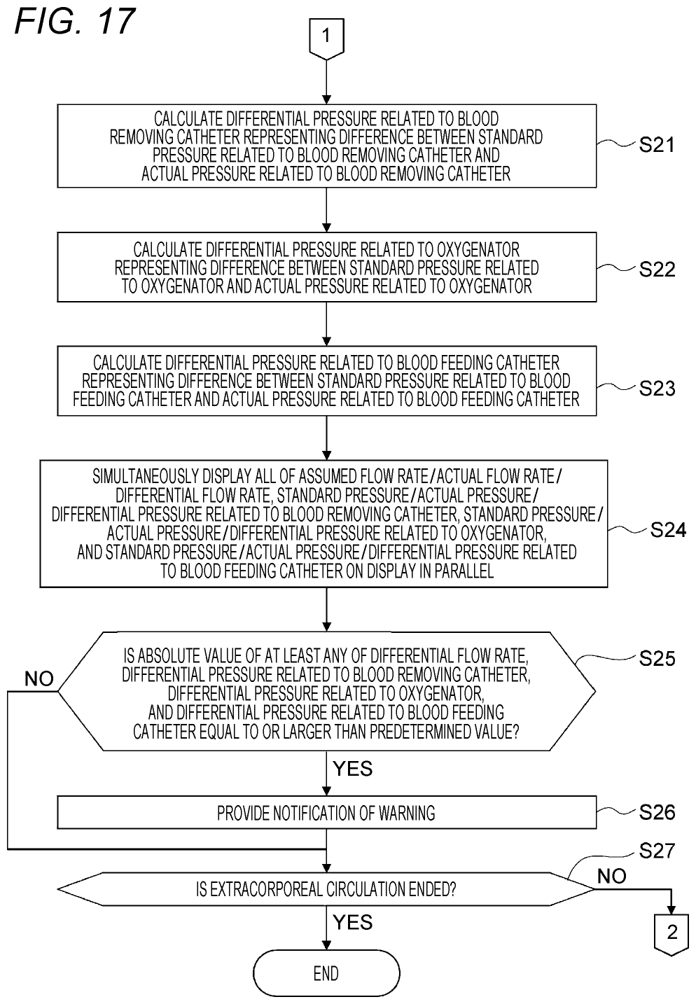

Next, a description will be given with reference to the drawings regarding control executed as the computer 51 of the extracorporeal circulation management device 10 according to the present embodiment reads the program 31 (extracorporeal circulation management program) stored in the storage unit 30. FIGS. 16 and 17 are flowcharts illustrating examples of the control executed by the computer of the extracorporeal circulation management device according to the present embodiment.

First, in step S11, the control unit 40 stores, for example, the expected flow rate 66, input by the operator or the like in response to the operator's operation or the like on the touch panel 52, in the expected flow rate storage unit 32. Alternatively, the control unit 40 stores the expected flow rate 66, calculated by the standard flow rate calculation unit 47 using patient information (for example, height, weight, and the like) input by the touch panel 52 in response to the operator's operation on the touch panel 52 and the standard information 334 related to the circulation circuit 1R stored in the standard information storage unit 33, in the expected flow rate storage unit 32. Subsequently, in step S12, the standard pressure calculation unit 431 related to the blood removing catheter 5 refers to the standard information 331 related to the blood removing catheter 5 stored in the standard information storage unit 33 and calculates the standard pressure 71 related to the blood removing catheter 5 based on the expected flow rate 66 stored in the expected flow rate storage unit 32.

Subsequently, in step S13, the standard pressure calculation unit 432 related to the oxygenator 2 refers to the standard information 332 related to the oxygenator 2 stored in the standard information storage unit 33 and calculates the standard pressure 74 related to the oxygenator 2 based on the expected flow rate 66 stored in the expected flow rate storage unit 32. Subsequently, in step S14, the standard pressure calculation unit 433 related to the blood feeding catheter 6 refers to the standard information 333 related to the blood feeding catheter 6 stored in the standard information storage unit 33 and calculates the standard pressure 77 related to the blood feeding catheter 6 based on the expected flow rate 66 stored in the expected flow rate storage unit 32.

Incidentally, the order of the processes described above with respect to steps S12 to S14 is not particularly limited. For example, the processes described above with respect to steps S12 to S14 may be executed at the same time. Alternatively, the processes described above with respect to steps S14, S13, and S12 may be executed in this order.

Subsequently, in step S15, the control unit 40 acquires the actual flow rate 67 from the flow rate measurement unit 24 through the communication unit 53. Then, in step S16, the actual pressure calculation unit 441 related to the blood removing catheter 5 acquires an actual pressure (actually measured value) from the first pressure sensor 21 through the communication unit 53, and calculates the actual pressure 72 related to the blood removing catheter 5 based on the actual pressure measured by the first pressure sensor 21 and the actual flow rate 67 acquired from the flow rate measurement unit 24. Specifically, the actual pressure calculation unit 441 related to the blood removing catheter 5 calculates a value, obtained by subtracting a blood pressure (for example, central venous pressure (CVP)) displayed on the biological monitor 15 from an actually measured value measured by the first pressure sensor 21, as a pressure loss that actually occurs in the blood removing catheter 5.

Then, in step S17, the actual pressure calculation unit 442 related to the oxygenator 2 acquires an actual pressure (actually measured value) from each of the second pressure sensor 22 and the third pressure sensor 23 through the communication unit 53, and calculates the actual pressure 75 related to the oxygenator 2 based on the actual pressure measured by the second pressure sensor 22, the actual pressure measured by the third pressure sensor 23, and the actual flow rate 67 acquired from the flow rate measurement unit 24. Specifically, the actual pressure calculation unit 442 related to the oxygenator 2 calculates a value, obtained by subtracting an actually measured value measured by the second pressure sensor 22 from an actually measured value measured by the third pressure sensor 23, as a pressure loss that actually occurs in the oxygenator 2.

Subsequently, in step S18, the actual pressure calculation unit 443 related to the blood feeding catheter 6 acquires the actual pressure (actually measured value) from the third pressure sensor 23 through the communication unit 53, and calculates the actual pressure 78 related to the blood feeding catheter 6 based on the actual pressure measured by the third pressure sensor 23 and the actual flow rate 67 acquired from the flow rate measurement unit 24. Specifically, the actual pressure calculation unit 443 related to the blood feeding catheter 6 calculates a value, obtained by subtracting the actually measured value measured by the third pressure sensor 23 from a blood pressure (for example, average blood pressure) displayed on the biological monitor 15, as a pressure loss that actually occurs in the blood feeding catheter 6.

Subsequently, in step S19, the differential flow rate calculation unit 45 calculates the differential flow rate 68 representing the difference between the expected flow rate 66 stored in the expected flow rate storage unit 32 and the actual flow rate 67 acquired from the flow rate measurement unit 24. Subsequently, in step S21, the differential pressure calculation unit 461 related to the blood removing catheter 5 calculates the differential pressure 73 related to the blood removing catheter 5 representing the difference between the standard pressure 71 related to the blood removing catheter 5 calculated by the process described above with respect to step S12 and the actual pressure 72 related to the blood removing catheter 5 calculated by the process described above with respect to step S16.

Subsequently, in step S22, the differential pressure calculation unit 462 related to the oxygenator 2 calculates the differential pressure 76 related to the oxygenator 2 representing the difference between the standard pressure 74 related to the oxygenator 2 calculated by the process described above with respect to step S13 and the actual pressure 75 related to the oxygenator 2 calculated by the process described above with respect to step S17. Subsequently, in step S23, the differential pressure calculation unit 463 related to the blood feeding catheter 6 calculates the differential pressure 79 related to the blood feeding catheter 6 representing the difference between the standard pressure 77 related to the blood feeding catheter 6 calculated by the process described above with respect to step S14 and the actual pressure 78 related to the blood feeding catheter 6 calculated by the process described above with respect to step S18.

Subsequently, in step S24, the display processing unit 41 displays all the expected flow rate 66, the actual flow rate 67, the differential flow rate 68, the standard pressure 71 related to the blood removing catheter 5, the actual pressure 72 related to the blood removing catheter 5, the differential pressure 73 related to the blood removing catheter 5, the standard pressure 74 related to the oxygenator 2, the actual pressure 75 related to the oxygenator 2, the differential pressure 76 related to the oxygenator 2, the standard pressure 77 related to the blood feeding catheter 6, the actual pressure 78 related to the blood feeding catheter 6, and the differential pressure 79 related to the blood feeding catheter 6 simultaneously and in parallel on the external monitor 16 as one display unit.

Subsequently, in step S25, the control unit 40 determines whether or not an absolute value of at least any of the differential flow rate 68, the differential pressure 73 related to the blood removing catheter 5, the differential pressure 76 related to the oxygenator 2, and the differential pressure 79 related to the blood feeding catheter 6 is equal to or larger than a predetermined value. When the absolute value of at least any of the differential flow rate 68, the differential pressure 73 related to the blood removing catheter 5, the differential pressure 76 related to the oxygenator 2, and the differential pressure 79 related to the blood feeding catheter 6 is equal to or larger than the predetermined value (step S25: YES), the notification processing unit 42 refers to the warning information stored in the warning information storage unit 35, and notifies the external monitor 16 of the warning information. As a result, the operator or the like can more easily grasp occurrence of a discrepancy or deviation between the expected flow rate 66 and the actual flow rate 67, and further grasp a location of the circulation circuit 1R where the discrepancy or deviation between the standard pressure 71, 74, or 77 and the actual pressure 72, 75, or 78 has occurred and more easily grasp that a cause of circulation failure exists near the location. Incidentally, an example of the warning information notification of which is provided by the notification processing unit 42 is the same as that described above with respect to FIG. 12.

On the other hand, when the absolute value of at least any of the differential flow rate 68, the differential pressure 73 related to the blood removing catheter 5, the differential pressure 76 related to the oxygenator 2, and the differential pressure 79 related to the blood feeding catheter 6 is not equal to or larger than the predetermined value (step S25: NO), the control unit 40 determines in step S27 whether or not the extracorporeal circulation is completed. In addition, in step S27 following step S26, the control unit 40 determines in step S27 whether or not the extracorporeal circulation is completed.

When the extracorporeal circulation is not completed (step S27: NO), the processing returns to step S15, and the control unit 40 acquires the actual flow rate 67 from the flow rate measurement unit 24 through the communication unit 53. On the other hand, when the extracorporeal circulation is completed (step S27: YES), the control unit 40 ends the execution of the program 31 related to the extracorporeal circulation.

According to the program 31 (extracorporeal circulation management program) of the present embodiment, the operator or the like can easily grasp a discrepancy or deviation between the expected flow rate 66 of blood in the circulation circuit 1R and the actual flow rate 67 of the blood actually flowing in the circulation circuit 1R by confirming the external monitor 16. In addition, the operator or the like can easily grasp a discrepancy or deviation between each of the standard pressures 71, 74, and 77 calculated based on the expected flow rate 66 and each of the actual pressures 72, 75, and 78 related to the respective devices (the blood removing catheter 5, the oxygenator 2, and the blood feeding catheter 6) by observing the external monitor 16. Therefore, when there is the discrepancy or deviation between the expected flow rate 66 and the actual flow rate 67, the operator or the like can easily grasp the occurrence of the discrepancy or deviation between the expected flow rate 66 and the actual flow rate 67 and grasp a location of the circulation circuit 1R where the discrepancy or deviation between the standard pressure 71, 74, or 77 and the actual pressure 72, 75, or 78 has occurred and easily grasp that a cause of circulation failure exists near the location by observing the external monitor 16. That is, the location of the circulation circuit 1R where the discrepancy or deviation between the standard pressure 71, 74, or 77 and the actual pressure 72, 75, or 78 has occurred corresponds to a portion where the pressure measurement unit 20 is provided or a portion of each device provided near the pressure measurement unit 20. In this manner, the operator or the like can easily grasp the cause of the circulation failure in the extracorporeal circulation. In addition, the same effects as those described above can be obtained with respect to FIGS. 13 and 14.

The embodiments of the present invention have been described above. However, the present invention is not limited to the above embodiments, and various modifications can be made within a range not departing from the scope of claims. The configurations of the above embodiments can be partially omitted or arbitrarily combined so as to be different from the above configurations.

What is claimed is:

1. An extracorporeal circulation system comprising;
a circulation circuit adapted to circulate blood taken out from a patient through a plurality of instrument elements and return the blood to a patient;
a flow rate measurement unit measuring an actual flow rate value of the blood flowing inside the circulation circuit;
a pressure measurement unit measuring an actual pressure value of the blood flowing inside the circulation circuit;
a display unit; and
an extracorporeal circulation management controller configured to 1) store an expected flow rate of the blood to be provided to the patient, 2) receive the actual flow rate from the flow rate measurement unit, 3) display the expected flow rate and the actual flow rate on the display unit, 4) calculate a standard pressure corresponding to at least one of the instrument elements based on the expected flow rate and on standard information which represents a relation between a blood flow rate and a pressure loss occurring in the at least one of the instrument elements, and 5) display the calculated standard pressure and the actual pressure value corresponding to the at least one of the instrument elements on the display unit.

2. The extracorporeal circulation system according to claim 1, wherein a differential flow rate representing a difference between the expected flow rate and the actual flow rate and a differential pressure representing a difference between the standard pressure and the actual pressure value are further displayed on the display unit.

3. The extracorporeal circulation system according to claim 2:
wherein the pressure measurement unit includes a plurality of pressure sensors, each pressure sensor measuring a respective actual pressure value between respective instrument elements;
wherein a plurality of respective standard pressures are calculated based on i) the expected flow rate by referring to a plurality of pieces of the standard information each representing a relation between the blood flow rate and the pressure loss occurring in each of the plurality of instrument elements, and ii) the actual pressures related to the plurality of instrument elements calculated based on a plurality of actual pressure values measured by the plurality of pressure sensors and the actual flow rate;
wherein a plurality of respective differential pressures are calculated, each representing a difference between a respective one of the plurality of standard pressures and a respective one of the actual pressure values related to a respective one of the plurality of instrument elements; and
wherein the plurality of standard pressure values, the plurality of actual pressure values, and the plurality of differential pressures are displayed on the display unit.

4. The extracorporeal circulation system according to claim 3, wherein the plurality of instrument elements includes:
a blood removing catheter which is partially inserted into the patient and guides the blood taken out from the patient;
a pump which is provided on a downstream side of the blood removing catheter, takes out the blood from the patient through the blood removing catheter and sends the blood to a downstream side of the pump;
an oxygenator which is provided on the downstream side of the pump and performs a gas exchange operation for the blood; and
a blood feeding catheter which is provided on a downstream side of the oxygenator and is partially inserted into the patient, and guides the blood that has passed through the oxygenator to the patient.

5. The extracorporeal circulation system according to claim 4:
wherein the plurality of pressure sensors includes a first pressure sensor provided in the circulation circuit between the blood removing catheter and the oxygenator and a second pressure sensor provided in the circulation circuit between the oxygenator and the blood feeding catheter; and
wherein the flow rate measurement unit is a flow rate sensor provided in the circulation circuit.

6. The extracorporeal circulation system according to claim 5 wherein the display simultaneously displays:
the expected flow rate;
the actual flow rate acquired by the flow rate sensor;
the differential flow rate;
a standard pressure related to the blood removing catheter calculated based on the expected flow rate by referring to the standard information related to the blood removing catheter which represents a relation between the blood flow rate and the pressure loss occurring in the blood removing catheter;
a standard pressure related to the oxygenator calculated based on the expected flow rate by referring to the standard information related to the oxygenator which represents a relation between the blood flow rate and the pressure loss occurring in the oxygenator;
a standard pressure related to the blood feeding catheter calculated based on the expected flow rate by referring to the standard information related to the blood feeding catheter which represents a relation between the blood flow rate and the pressure loss occurring in the blood feeding catheter;
an actual pressure related to the blood removing catheter calculated based on an actual pressure measured by the first pressure sensor and the actual flow rate;
an actual pressure related to the oxygenator calculated based on the actual pressure measured by the first pressure sensor, an actual pressure measured by the second pressure sensor, and the actual flow rate;
an actual pressure related to the blood feeding catheter calculated based on the actual pressure measured by the second pressure sensor and the actual flow rate;
a differential pressure related to the blood removing catheter which represents a difference between the standard pressure related to the blood removing catheter and the actual pressure related to the blood removing catheter;
a differential pressure related to the oxygenator which represents a difference between the standard pressure related to the oxygenator and the actual pressure related to the oxygenator; and
a differential pressure related to the blood feeding catheter which represents a difference between the standard pressure related to the blood feeding catheter and the actual pressure related to the blood feeding catheter.

7. The extracorporeal circulation system of claim 2, wherein notification of a warning is provided when an absolute value of at least one of the differential flow rate and the differential pressure exceeds a predetermined value.

8. An extracorporeal circulation system circulating blood of a patient, comprising:
a display unit that displays various types of information;
a circulation circuit including:
a blood removing catheter which is partially inserted into the patient and guides the blood taken out from the patient;
a pump which is provided on a downstream side of the blood removing catheter, takes out the blood from the patient through the blood removing catheter, and sends the blood to a downstream side of the pump;
an oxygenator which is provided on the downstream side of the pump and performs a gas exchange operation for the blood;
a blood feeding catheter which is provided on ae downstream side of the oxygenator, is partially inserted into the patient, and guides the blood that has passed through the oxygenator to the patient;
a flow rate measurement unit measuring an actual flow rate value of the blood flowing inside the circulation circuit;
a pressure measurement unit measuring an actual pressure value of the blood flowing inside the circulation circuit;
an extracorporeal circulation management controller configured to 1) store an expected flow rate of the blood to be provided to the patient, 2) receive the actual flow rate from the flow rate measurement unit, 3) calculate a differential flow rate representing a difference between the expected flow rate and the actual flow rate, 4) calculate a standard pressure corresponding to each of the blood removing catheter, the pump, the oxygenator, and the blood feeding catheter based on the expected flow rate and on standard information which represents a relation between a blood flow rate and a pressure loss occurring in each of the blood removing catheter, the pump, the oxygenator, and the blood feeding catheter, 5) calculate a respective differential pressure representing a difference between the standard pressures corresponding to each of the blood removing catheter, the pump, the oxygenator, and the blood feeding catheter and respective actual pressure values, and 6) display the expected flow rate, the actual flow rate, the calculated standard pressures, the actual pressure values, and the differential pressures on the display unit.

9. The extracorporeal circulation system of claim 8, wherein the extracorporeal circulation management controller is further configured to display a notification of a warning on the display unit when an absolute value of at least one of the differential flow rate and the differential pressures exceeds a respective predetermined value.

10. A non-transitory computer readable media having a extracorporeal circulation management program, executed by a computer of an extracorporeal circulation management device that manages an extracorporeal circulation device which extracorporeally circulates blood using a circulation circuit, the extracorporeal circulation management program causing the computer to execute operations comprising:
displaying an expected flow rate, which is expected in advance as an expected value of a flow rate of the blood flowing inside the circulation circuit, on a display unit;
displaying an actual flow rate, which is measured by a flow rate measurement unit as an actually measured value of the flow rate of the blood flowing inside the circulation circuit, on the display unit; and
displaying a standard pressure calculated based on the expected flow rate by referring to standard information, which represents a relation between the blood flow rate and a pressure loss occurring in a device provided in the circulation circuit and is stored in a storage unit;
displaying an actual pressure related to the device, which is calculated based on an actual pressure measured by a pressure measurement unit as an actually measured value of a pressure of the blood flowing inside the circulation circuit and the actual flow rate, on the display unit.

11. The non-transitory computer readable media of claim 10 wherein the operations further comprise:
displaying a differential flow rate representing a difference between the expected flow rate and the actual flow rate on the display unit; and
displaying a differential pressure representing a difference between the standard pressure and the actual pressure value on the display unit.

12. The non-transitory computer readable media of claim 11 wherein the operations further comprise:
displaying a notification of a warning on the display unit when an absolute value of at least one of the differential flow rate and the differential pressure exceeds a respective predetermined value.
